(19) United States
(12) Patent Application Publication
(10) Pub. No.: US 2007/0258993 A1
(43) Pub. Date: Nov. 8, 2007

(54) Dna-Carrier Conjugate

(75) Inventors: Vasso Apostolopoulos, St Albans (AU); Geoffrey Pietersz, Greensborough (AU); Ian McKenzie, Point Lonsdale (AU); Choon Kit Tang, Mill Park (AU)

Correspondence Address: ROTHWELL, FIGG, ERNST & MANBECK, P.C., 1425 K STREET, N.W., SUITE 800, WASHINGTON, DC, 20005, US

(73) Assignee: The Austin Research Institute, Heidelberg (AU)

(21) Appl. No.: 10/579,229

(22) PCT Filed: Nov. 12, 2004
(86) PCT No.: PCT/AU04/01564

§ 371 Date: Apr. 6, 2007

(30) Foreign Application Priority Data
Nov. 12, 2003 (AU) 2003906217

(51) Int. Cl. A61K 39/385; A61P 31/12; C07H 21/02

(52) U.S. Cl. 424/193.1; 536/23.5

(57) ABSTRACT

A cell-specific delivery method of genetic material for the purposes of providing polynucleotide- or oligonucleotide-based genetic vaccines or a means for gene therapy. The delivery method involves the use of a compound comprising a conjugate of a polynucleotide or oligonucleotide molecule, a carrier comprising at least one aldehyde group and, optionally, a suitable linker molecule.

### FIELD OF THE INVENTION

This invention relates to the cell-specific delivery of genetic material for the purposes of providing polynucleotide- or oligonucleotide-based genetic vaccines or a means for gene therapy. The invention particularly relates to a compound comprising a conjugate of a polynucleotide or oligonucleotide molecule, a carrier comprising at least one aldehyde group and, optionally, a suitable linker molecule.

### BACKGROUND OF THE INVENTION

The development of an inexpensive, safe, readily applicable gene transfer system, efficient in transgene expression and able to target a cell type of choice in vivo, is crucial to the success of both polynucleotide-based genetic vaccines (eg DNA vaccines) and gene therapy.

Delivery of genetic material capable of expressing a particular protein or peptide has many advantages over the delivery of the protein/peptide itself. That is, with live or attenuated protein vaccination, there is always a risk that a patient will inadvertently be given the infectious agent itself, whereas with genetic or DNA vaccination this risk is absent. Further, unlike peptide-based vaccines, which allow only a limited number of epitopes, DNA vaccines may encode multiple epitopes (ie a so-called polytope vaccines). Moreover, genetic vaccines are generally easy and cheap to produce in large quantities and there is usually no need for special handling and storage conditions.

Various methods for delivering genetic material to cells for intracellular expression have been investigated. These include direct delivery of genetic material, viral delivery systems and non-viral carrier systems.

Direct deliveries of “naked” DNA have tended to yield only fair immune responses in preclinical and clinical studies. A particular example of this kind of delivery is the “Gene Gun” technology, which has been shown to achieve considerable efficiency in delivering the DNA into the epidermis of a subject, but has the disadvantage in that the delivered DNA is not specifically directed to any particular cell type, and nor is the DNA protected in any way from degradation through the action of nudeases. Further, the associated costs with Gene Gun technology are typically high. The majority of gene therapy clinical trials performed so far have utilised viruses (eg adenoviruses and retroviruses) as carriers. Other viral carriers used in clinical trials have included adeno-associated and Herpes viruses. Such viral carriers or vectors have been popular choices due to their advantages of having a high transfection rate and also the high chance of stable and long-term expression of the delivered gene in the target cell's genome. However, there are also many disadvantages associated with using viral vectors for gene delivery. The most important of these is the safety concern associated with introducing viruses into the body. That is, there is the possibility that a viral vector will elicit a potent immune reaction in the subject, and indeed, this has been thought to be the cause of death of some patients in clinical gene therapy trials. Moreover, there is also a risk that the virus will evolve and mutate to give rise to a new viral disease, and more importantly, induce malignant transformation in the subject.

Non-viral vectors are therefore widely considered to present a safer option as a carrier of genetic material. Various forms of non-viral vectors have been designed, for example cationic liposomes, cationic lipids, microparticles and receptor-mediated gene transfer ligands. All of these aim to transfer genetic material with essentially no side effects and facilitate transfer to specific cell types. Unfortunately though, most of them do not presently offer the high transfection rate of viral transfer, which has evolved efficient mechanisms to transfer genetic material into cells and protect the genetic material from degradation by intracellular enzymes.

However, a further example of a non-viral carrier system, namely receptor-mediated gene therapy has demonstrated promising results. This technique achieves cell-specific gene delivery by using ligands targeted to cell surface receptors conjugated with the genetic material to be delivered, and unlike lipofection (using cationic liposomes coupled with negatively charged DNA), which is a classical non-viral carrier system that often causes systemic side effects, receptor-mediated gene therapy appears to be safe and cell-specific. To date, there have been several receptor-mediated gene delivery systems developed; among the more popular ones are those targeting transferrin, neurotensin and the mannose receptor (Erbacher, P et al, 1996; Ferkol, T et al, 1996; and Diebold, S S et al, 1999a). Generally, the transfer of genetic material by such systems involves: (1) conjugation of DNA with a receptor-specific ligand followed by DNA condensation; (2) binding of the DNA/ligand complex to the cell surface receptor; (3) internalisation of the complex together with the receptor by an endosome; (4) release of the complex from the endosome; (5) translocation of the DNA into the nucleus; and (6) expression of the delivered DNA. In this process, the DNA condensation is often vital to the successful transfer of the genetic material (Liu, G et al, 2001). This is usually mediated by a polycation linker that links the receptor-specific ligand to the DNA; polycation ligands such as poly-L-lysine (PLL), polyethylenimine (PEI) and cationic lipids are commonly used to condense the negatively charged DNA. Also vital for efficient transfer by receptor-mediated gene transfer techniques is the minimisation of endosomal degradation of DNA. In this regard, PEI as a linker has previously been shown to be effective in preventing endosomal degradation (Boussif, O et al, 1995), however as toxicity and transfection efficiencies vary greatly depending on the type and size of the polycation linker, optimisation studies need to be addressed.

The mannose receptor (MR) is a multilectin cell surface receptor, mainly found on macrophages, dendritic cells and some endothelial cells which bind to various carbohydrate residues (eg mannose). The use of mannose to target MR has been widely studied as a possible basis for a non-viral carrier system for delivery of genetic material to a subject. In particular, mannose has been previously investigated for the delivery of genetic material to airway cells expressing MR such as airway epithelial cells (Fajac, I et al, 2002), dendritic cells (Diebold, S S et al, 1999a) and macrophages (Ferkol, T et al, 1996) affected by cystic fibrosis. The complexes used in those investigations comprised either mannose-PLL or mannose-PEI and DNA. Upon binding to the receptor, the complexes were endocytosed and efficiently processed by the cell, ultimately leading to presentation of expressed antigen to effector cells. Also, mannosylated cationic liposomes have been investigated and shown to facilitate mannose receptor gene transfer into macrophages (Diebold, S S et al, 1999b; and Sato, A et al, 2001), and further, plasmid DNA encoding luciferase (pCMV-Luc) complexed with mannosylated cationic liposomes have been shown to achieve significantly higher transfection of mouse peritoneal macrophages than non-mannosylated cationic liposomes (Diebold, S S et al, 1999b; and Sato, A et al, 2001). Moreover, it has recently been shown that biodegradable nanoparticles (ie warm oil-in-water microemulsion particles) coated with DNA and mannan (a polysaccharide of mannose), can be introduced to macrophage cells and, notably, a 50% higher uptake was achieved by the mannan-coated particles relative to non-mannan-coated particles (Cui, Z and Mumper, R J, 2002a; Cui, Z and Mumper, R J, 2002b; and Cui, Z et al, 2003). Taken as a whole, these studies indicate that mannose can function as an effective carrier of genetic material to mannose receptor-positive cells within in a subject.

In previous work by the applicant, mannan, in its oxidised form (ie with one or more aldehyde groups), conjugated to a tumour associated antigen, MUC1 fusion protein (MUC1-FP), was used to target the antigen to macrophages and dendritic cells (DCs). In particular, it was found that when injected in its oxidised form (using sodium periodate), mannan induced a strong CD8⁺ T cell response but weak antibody responses (Lofthouse, S A et al, 1997; McKenzie, I F et al., 1998; and Apostolopoulos, V et al, 2000). In contrast, mice injected with reduced mannan (ie oxidised mannan treated with sodium borohydride to reduce aldehyde groups to hydroxyl groups) provoked weak CD8⁺ T cell responses but strong antibody responses, thus indicating that the reduced mannan induced a CD4⁺ T cell response (Apostolopoulos, V et al, 1995; and Apostolopoulos, V et al, 1996). It was also demonstrated that oxidised mannan appeared to help prevent MUC1 fusion protein (MUC1-FP) from degradation by facilitating escape of the protein from the endosome before it fuses to lysosomes containing degradative enzymes.

In view of this previous work, the applicant decided to investigate the possibility of using a receptor-specific ligand as a means for achieving cell-specific delivery of genetic materials, wherein the ligand includes at least one aldehyde group to facilitate endosomal release. Using DNA conjugated to either oxidised mannan (ie mannan with multiple exposed aldehyde groups) and reduced mannan (wherein aldehyde groups are reduced to hydroxyl groups) through a polycation linker, it was surprisingly found that the use of the oxidised mannan conjugates resulted in a primarily CD8⁺ type immune response, whereas the reduced mannan conjugates resulted in a primarily CD4⁺ type immune response. Further, it was also surprisingly found that the use of either oxidised or reduced mannan conjugates, at higher doses, induced both a strong CD8⁺ type immune response and a strong CD4⁺ type immune response. Thus, oxidised mannan conjugates and reduced mannan conjugates can be used to tailor the immune response to a given antigen to either a CD4⁺ T cell response or a CD8⁺ T cell response or both.

### SUMMARY OF THE INVENTION

In a first aspect, the present invention provides a compound comprising a conjugate of;

(i) a polynucleotide or oligonucleotide molecule;

(ii) a carrier comprising at least one aldehyde group; and, optionally,

(iii) a suitable linker molecule conjugating said polynucleotide or oligonucleotide with said carrier.

Preferably, the polynucleotide or oligonucleotide comprises a nucleotide sequence encoding a protein or peptide of interest (eg an antigen or epitope(s)), but may also be antisense or catalytic RNA (eg a ribozyme) targeted against a gene expressed in a target cell. The polynucleotide or oligonucleotide molecule may also constitute a small interfering RNA (siRNA) targeted against a gene expressed in a target cell.

Preferably, the carrier comprises a plurality of aldehyde groups (eg in the range of 20 to 750 aldehyde groups). More preferably, the carrier is a carbohydrate polymer comprising a plurality of aldehyde groups (eg in the range of 200 to 400 aldehyde groups) such as oxidised mannose.

Preferably, the compound comprises a suitable linker molecule conjugating the polynucleotide or olignucleotide molecule to the carrier. Suitable linker molecules include polycation linkers such as PLL, PEI, dendrimers and cationic lipids.

In a second aspect, the present invention provides a method for cell-specific delivery of a polynucleotide or oligonucleotide molecule to a target cell(s) of a subject, said method comprising:
providing a compound comprising a conjugate of;
   (i) a polynucleotide or oligonucleotide molecule;
   (ii) a carrier comprising at least one aldehyde group; and, optionally,
   (iii) a suitable linker molecule conjugating said polynucleotide or oligonucleotide with said carrier; and
administering said compound to said subject.

In a third aspect, the present invention provides a method for inducing an immune response to an antigen or epitope(s), wherein said immune response is primarily a CD8′ type of immune response, said method comprising:
providing a compound comprising a conjugate of;
   (i) a polynucleotide or oligonucleotide molecule comprising a nucleotide sequence encoding an antigen or epitope(s);
   (ii) a carrier comprising at least one aldehyde group; and, optionally,
   (iii) a suitable linker molecule conjugating said polynucleotide or oligonucleotide with said carrier; and
administering said compound to said subject in an amount to induce a primarily CD8⁺ type of immune response to said antigen or epitope(s).

In a fourth aspect, the present invention provides a method for inducing an immune response to an antigen or epitope(s), wherein said immune response is primarily a CD8⁺ type of immune response, said method comprising:
providing a compound comprising a conjugate of;
   (i) a polynucleotide or oligonucleotide-molecule comprising a nucleotide sequence encoding an antigen or epitope(s);
   (ii) a carrier comprising oxidised mannan; and, optionally,
   (iii) a suitable linker molecule conjugating said polynucleotide or oligonucleotide with said carrier; and
administering said compound to said subject in an amount to induce a primarily CD8⁺ type of immune response to said antigen or epitope(s).

In a fifth aspect, the present invention provides a method for inducing an immune response to an antigen or epitope(s), wherein said immune response is primarily a CD4⁺ type of immune response, said method comprising:
providing a compound comprising a conjugate of;
   (i) a polynucleotide or oligonucleotide molecule comprising a nucleotide sequence encoding an antigen or epitope(s);
   (ii) a carrier comprising reduced mannan; and, optionally,
   (iii) a suitable linker molecule conjugating said polynucleotide or oligonucleotide with said carrier; and
administering said compound to said subject in an amount to induce a primarily CD4⁺ type of immune response to said antigen.

Finally, in a sixth aspect, the present invention provides a compound comprising a conjugate of;

(i) a polynucleotide or oligonucleotide molecule;

(ii) a carrier comprising reduced mannan; and, optionally,

(iii) a suitable linker molecule conjugating said polynucleotide or oligonucleotide with said carrier.

### BRIEF DESCRIPTION OF THE FIGURES

FIG. 1 shows plasmids pEGFP-C1 (A) and sOVA-C1 (B) used in the examples herein.

FIG. 2 shows the efficiency of DNA complexation analysed by (A) 1% agarose gel electrophoresis of OxMan-PLL-DNA conjugated at various NaCl concentration (0, 0.9, 1 and 1.1 M) and PLL:DNA nucleotide molar ratio (r=0, 0.25, 0.5, 0.75 and 1), (B) 0.6% agarose gel electrophoresis (1 hour at 100V) of OxMan-PLL-DNA conjugated at varying DNA:PLL nucleotide molar ratios (r=0, 0.1, 0.25, 0.4, 0.5, 0.75, 1, 2, 3, 4, 5 and 10) and at 0.7 M NaCl, and (C) 0.6% agarose gel electrophoresis (1 hour at 100V) of OxMan-PLL-DNA conjugated at a PLL:DNA nucleotide molar ratios of r=0.4 and 2 and at various NaCl concentration (0, 0.3, 0.7, 1, 2 and 3 M).

FIG. 3 provides graphs showing the percentage binding/uptake of OxMan-FITC, RedMan-FITC and mannose-PLL-FITC by dendritic cells (DCs) or macrophages, incubated at various conjugate doses and times, by flow cytometry. These experiments were done at 37° c., thus binding or uptake or both is observed.

FIG. 4 provides graphs showing the percentage of expression of eGFP conjugated to OxMan-PLL (o-pll), RedMan-PLL (r-pll), mannose-PLL (m-pll), mannose-PEI (m-pei), DNA alone (with 700 mM and 900 mM NaCl), PLL, PEI, Fugene and nothing added (neg), (A) DC cultures, (B) macrophages, and (C) J774 macrophage cell lines. Different doses of carriers added (150, 100 and 50 μg) of OxMan-PLL and RedMan-PLL. The PLL:DNA nucleotide ratio used for mannose-PLL and mannose-PEI were r+1 and r+0.75. Errors in determining expression is approximately 10%.

FIG. 5 provides representative FACs profile of DC cultures incubated with OxMan-PLL-DNA, RedMan-PLL-DNA, mannose-PLL-DNA and mannose-PEI-DNA. The level of toxicity each carrier has is demonstrated by the percentage of dead cells (R3) and alive cells (r1 and R2) in PI vs FSC plot.

FIG. 6 provides graphical results showing the levels of toxic effect of OxMan-PLL, RedMan-PLL, mannose-PLL, mannose-PEI, PLL and PEI on DCs. Cell viability is demonstrated by [³H] uptake in cpm. Nothing added is shown (11077 cpm).

FIG. 7 shows the results of proliferation assays in a pilot in vivo study. T cells of mice vaccinated with DNA alone, PLL-DNA, OxMan-PLL-DNA and RedMan-PLL-DNA were stimulated with whole ovalbumin peptide, OVA CD8 epitope peptide (SIINFEKL (SEQ ID NO: 1)), OVA CD4 epitope peptide (ISQAVHAAHAEINEAGR (SEQ ID NO: 2)). Polyclonal mitogen, ConA was used as a positive control and no peptide (no-stim) was used as a negative control. Individual mice (3 mice/group) are shown.

FIG. 8 shows the results of ELISPOT assays for IFN-γ secretion. Spleen cells from immunised mice were pulsed with whole ovalbumin peptide, OVA CD8 epitope peptide (SINFEKL (SEQ ID NO: 1)), OVA CD4 epitope peptide (ISQAVHAAHAEINEAGR (SEQ ID NO: 2)). Polyclonal mitogen, ConA was used a s a positive control and no peptide added as negative control. Individual mice (3 mice/group) are shown.

FIG. 9 shows antibody responses to whole ovalbumin as assessed by ELISA. Sera from individual mice were tested for total immunoglobulin cintent reactive against ovalbumin before injections (prebleeds) and after 1, 2 and 3 injections of various carriers conjugated to OVA. Antibody levels (1/50 to 1/102400). The magenta line within each group represents antibody level in naïve mice.

FIG. 10 shows results obtained from C57BL/6 mice immunised on day 0 and 14 with 10 or 50 μg DNA linked to the various carriers and 10-14 days after final injection, mouse splenocytes were isolated and proliferation to ovalbumin, ovalbumin CD4 or CD8 epitopes were measured on days 1-5. ConA was used as a positive control and nothing was used as a negative control. (A) DNA 10 μg, (B) DNA 50 μg, (C) DNA-PLL 10 μg, (D) DNA-PLL 50 μg, (E) RedMan-PLL-DNA 10 μg, (F) RedMan-PLL-DNA 50 μg, (G) OxMan-PLL-DNA 10 μg, (H) OxMan-PLL-DNA 50 μg, (I) mannose-PLL-DNA 10 μg, (j) mannose-PLL-DNA 50 μg. Error bars depict standard error of the mean.

FIG. 11 shows results obtained from C57BL/6 mice immunised on day 0 and 14 with 10 or 50 μg DNA linked to the various carriers and 10-14 days after final injection, mouse splenocytes were isolated and IFNγ and IL4 secretion to ovalbumin protein, CD4 or CD8 epitopes were measured. ConA was used as a positive control and nothing was used as a negative control. (A) DNA 10 μg, (B) DNA 50 μg, (C) DNA-PLL 10 μg, (D) DNA-PLL 50 μg, (E) RedMan-PLL-DNA 10 μg, (F) RedMan-PLL-DNA 50 μg, (G) OxMan-PLL-DNA 10 μg, (h) OxMan-PLL-DNA 50 μg, (I) mannose-PLL-DNA 10 μg, (j) mannose-PLL-DNA 50 μg. Error bars depict standard error of the mean.

FIG. 12 shows results obtained from C57BL/6 mice immunised on day 0 and 14 with 10 or 50 μg DNA linked to the various carriers and 14 days after final injection, challenged with EG7 tumour cells (OVA-EL4). Tumour growth was monitored by measuring the two perpendicular diameters using a calliper. (A) PBS, (B) PLL-DNA (10 μg), (C) PLL-DNA (50 μg), (D) RedMan-PLL-DNA (10 μg), (E) RedMan-PLL-DNA (50 μg), (F) OxMan-PLL-DNA (10 μg), (G) OxMan-PLL-DNA (50 μg).

### DETAILED DESCRIPTION OF THE INVENTION

In its broadest aspect, the present invention provides a compound comprising a conjugate of:

(i) a polynucleotide or oligonucleotide molecule;

(ii) a carrier comprising at least one aldehyde group; and, optionally,

(iii) a suitable linker molecule conjugating said polynucleotide or oligonucleotide with said carrier.

The polynucleotide or oligonucleotide molecule included in the compound may be single-stranded or double-stranded DNA (eg cDNA and genomic DNA) or RNA. Oligonucleotides (including peptidenucleic acids and phosphothioate-modified nucleic acids) suitable for inclusion in the compound may be in the range of 5 to 50 bases in length, whereas polynucleotides suitable for inclusion in the compound may be in the range of 50 bases to 10 kilobases, more preferably, 1 to 6 kilobases.

Preferably, the polynucleotide or oligonucleotide molecule comprises an expression cassette comprising a suitable promoter sequence operably linked to a nucleotide sequence encoding a protein(s) or peptide(s) of interest such as an antigen or one or more epitopes (eg a polytope peptide) which may or may not be fused to a suitable fusion partner (eg glutathione-S-transferase) so as to form the basis of a genetic vaccine. The antigen or epitope(s) may be associated with an infectious disease, cancer, autoimmune condition or inflammation.

Particular preferred examples of encoded antigens associated with infectious diseases include viral antigens such as the hepatitis B virus (HBV) envelope Ag pre S2 protein, the hepatitis C virus (HCV) core antigen, HIV-gp120/160 envelope glycoprotein, influenza nucleoprotein, rabies virus G protein, respiratory syncyticial virus (RSV) F and G proteins, Epstein Barr virus (EBV) gp340 and nucleoantigen 3A, Varicella zoster virus IE62 and gpI, Rubella virus capsid protein, human rhinovirus (HRV) capsid protein, papillomavirus peptides from oncogene E6 and E7, and antigens from various infectious microorganisms including the Plasmodium falciparum circumsporozoite protein, Leishmania major surface glycoprotein (gp63), Bordetella pertussis surface protein, Streptococcus M protein, Mycobacterium tuberculosis 38 kDa lipoprotein or Ag85, Neisseria meningitidis class I outerprotein, chlamydia trachomatis surface protein and Listeria surface protein.

Particular preferred examples of encoded antigens associated with cancer include cancer-associated antigens such as the human mucin MUC1-MUC19 antigens (Marjolijn, J L et al, 1990; Crocker, G and Price, M R, 1987; Apostolopoulos, V et al, 1993; and Bobek, L A et al, 1993), carcinoembryonic antigen (CEA), survivin, Cripto-1, telomerase, claudin 7, Her2/Neu, Pim-1, p53, NM23, prostate specific antigen (PSA) and melanoma-specific antigens (eg MAGE series antigens).

Alternatively, the polynucleotide or oligonucleotide molecule comprises a nucleotide sequence encoding a protein or peptide of interest such as an enzyme, receptor or hormone which may be lacking or defective in a disease or condition so as to provide the basis for a gene therapy agent. For example, for treatment of cystic fibrosis, the compound may comprise a polynucleotide molecule encoding the cystic fibrosis transmembrane regulator (CFIR) protein.

The polynucleotide or oligonucleotide molecule may also be antisense or catalytic RNA (eg a ribozyme) targeted against a gene expressed in a target cell, or might otherwise constitute a small interfering RNA (siRNA) targeted against a gene expressed in a target cell (ie as described in Akkina, R et al, 2003, the entire disclosure of which is to be regarded as incorporated herein by reference).

Preferably, the carrier comprises a plurality of aldehyde groups ranging in number from 20 to 750, more preferably 100 to 500, most preferably 200 to 400. The carrier may be any suitable ligand which is recognised by a cell-surface receptor and, following binding to the receptor, can be endocytosed. Accordingly, the carrier may be a suitable ligand selected from hormones, enzymes, cytokines (eg an interferon, interleukin or colony stimulating factor) and, more preferably, carbohydrate polymers. If necessary, aldehyde groups may be introduced to the suitable ligand by reacting the ligand with any suitable oxidising agent (eg sodium periodate, Tollen's reagent and bromine water). Most preferably, the carrier included in the compound is an oxidised carbohydrate polymer, in particular oxidised mannan.

While not wishing to be bound by theory, it is believed that the at least one aldehyde group present on the carrier prevents degradation of the polynucleotide or oligonucleotide molecule upon endocytosis of the compound into a target cell, by bringing about the release of the polynucleotide or oligonucleotide molecule from the formed endosome into the cytoplasm before the endosome fuses with a lysosome containing degradative enzymes. From the cytoplasm, the polynucleotide or oligonucleotide molecule may be translocated into the nucleus where it may, for example, be replicated or transcribed. It is therefore considered that the compound of the present invention provides a means for efficient cell-specific delivery of genetic material to a target cell(s) of a subject and may, therefore, be well suited for application to polynucleotide-based genetic vaccines and gene therapy.

Preferably, the compound comprises a suitable linker molecule conjugating the polynucleotide or oligonucleotide molecule to the carrier. Suitable linker molecules include cross-linking agents such as biotin/streptavidin, oligopeptides, and polycation linkers such as PLL, PEI and cationic lipids. Such polycation linkers assist in condensing the polynucleotide or oligonucleotide molecule in the compound.

The compound of the present invention appears to be substantially non-toxic on administration to a subject and as a consequence is well tolerated by the subject.

As used herein, the term “conjugate” refers to the linkage of the polynucleotide or oligonucleotide molecule with the carrier by either covalent bonding or non-covalent bonding. Where a polycation linker is used to conjugate the polynucleotide or oligonucleotide molecule with the carrier, the linkage is made by non-covalent, electrostatic attraction of the positive charge of the polycation linker and the negative charge of the polynucleotide or oligonucleotide molecule.

The term “oxidised mannan” as used herein refers to mannan comprising at least one aldehyde group.

As indicated above, wherein the carrier comprises a suitable ligand which is recognised by a cell surface receptor, the compound of the present invention may be used for the cell-specific delivery of the polynucleotide or oligonucleotide molecule included in the compound to a target cell(s) of a subject. For example, by using oxidised mannan as the carrier, the polynucleotide or oligonucleotide molecule may be delivered to cells including the cell surface mannose receptor (MR) such as dendritic cells (DCs) and macrophages.

Thus, the present invention also provides a method for cell-specific delivery of a polynucleotide or oligonucleotide molecule to a target cell(s) of a subject, said method comprising:
providing a compound comprising a conjugate of;
   (i) a polynucleotide or oligonucleotide molecule;
   (ii) a carrier comprising at least one aldehyde group; and, optionally,
   (iii) a suitable linker molecule conjugating said polynucleotide or oligonucleotide with said carrier; and
administering said compound to said subject.

The compound may be formulated with any pharmaceutically-acceptable delivery vehicle or adjuvant for administration to the subject. Administration may be by any suitable mode including, for example, intramuscular injection, intravenous administration, nasal administration via an aerosol spray, and oral administration.

The amount of the compound that may be administered may vary upon a number of factors including the immune status of the subject and the severity of any disease or condition being treated. However, by way of example, the compound may be administered to a subject in an amount ranging from 1 to 10,000 μg/kg body weight, more preferably within the range of 10 to 100 μg/kg body weight.

It has been found that when oxidised or reduced mannan is used as a carrier for a conjugated polynucleotide or oligonucleotide molecule, variation in the type of immune response to an antigen induced in a subject can be achieved. It is anticipated that a similar effect can be achieved with other types of carbohydrate polymers.

Thus, the present invention provides a method for inducing an immune response to an antigen or epitope(s), wherein said immune response is primarily a CD8 type of immune response, said method comprising:
providing a compound comprising a conjugate of;
   (i) a polynucleotide or oligonucleotide molecule comprising a nucleotide sequence encoding an antigen or epitope(s);
   (ii) a carrier comprising at least one aldehyde group; and, optionally,
   (iii) a suitable linker molecule conjugating said polynucleotide or oligonucleotide with said carrier; and
administering said compound to said subject in an amount to induce a primarily CD8⁺ type of immune response to said antigen or epitope(s).

Preferably, the present invention provides a method for inducing an immune response to an antigen or epitope(s), wherein said immune response is primarily a CD8⁺ type of immune response, said method comprising:
providing a compound comprising a conjugate of;
   (i) a polynucleotide or oligonucleotide molecule comprising a nucleotide sequence encoding an antigen or epitope(s);
   (ii) a carrier comprising oxidised mannan; and, optionally,
   (iii) a suitable linker molecule conjugating said polynucleotide or oligonucleotide with said carrier; and
administering said compound to said subject in an amount to induce a primarily CD8⁺ type immune response to said antigen or epitope(s).

Further, the present invention provides a method for inducing an immune response to an antigen or epitope(s), wherein said immune response is primarily a CD4⁺ type of immune response, said method comprising:
providing a compound comprising a conjugate of;
   (i) a polynucleotide or oligonucleotide molecule comprising a nucleotide sequence encoding an antigen or epitope(s);
   (ii) a carrier comprising reduced mannan; and, optionally,
   (iii) a suitable linker molecule conjugating said polynucleotide or oligonucleotide with said carrier; and
administering said compound to said subject in an amount to induce a primarily CD4⁺ type of immune response.

Moreover, the present invention provides a compound comprising a conjugate of;

(i) a polynucleotide or oligonucleotide molecule;

(ii) a carrier comprising reduced mannan; and, optionally,

(iii) a suitable linker molecule conjugating said polynucleotide or oligonucleotide with said carrier.

As used herein, the term “reduced mannan” refers to mannan having no aldehyde groups and at least one hydroxyl group.

In the methods of the present invention for inducing an immune response to an antigen or epitope(s), the compound may be formulated with any pharmaceutically-acceptable delivery vehicle or adjuvant for administration to the subject. Administration may be by any suitable mode including, for example, intramuscular injection, intravenous administration, nasal administration via an aerosol spray, and oral administration.

In the methods of the present invention for inducing a CD8⁺ type or CD4⁺ type immune response to an antigen, the amount of the compound that may be administered may need to be selected to ensure that the desired type of immune response is primarily induced. By way of example, using a compound comprising oxidised mannan, the amount of the compound administered to induce a primarily CD8⁺ type immune response to an antigen may be deduced by routine trial—the amount would typically provide a dose of the polynucleotide or oligonucleotide molecule in the range of about 1 to 10000 μg, more preferably 100 to 1000 μg. Similarly, using a compound comprising reduced mannan, the amount of the compound administered to induce a primarily CD4⁺ immune response to an antigen may be deduced by routine trial—the amount would typically provide a dose of the polynucleotide or oligonucleotide molecule in the range of about 1 to 10000 μg, more preferably 100 to 1000 μg.

In order that the nature of the present invention may be more clearly understood, preferred forms thereof will now be described with reference to the following non-limiting examples.

The following abbreviations are used in the examples:
ABTS 2,2-Azino-di-[3-ethylbenzthiozoline sulphonate]
ACK lysis buffer Buffered ammonium chloride lysis solution
BSA Bovine Serum Albumin
DCs Dendritic cells
eGFP Enhanced green fluorescence protein
ELISA Enzyme-Linked Immunosorbent Assay
ELISPOT Enzyme linked immunospot
FACS Fluorescence activated cell sorter
FITC Fluorescein isothiocyanate
kDa Kilodaltons
mPBS Mouse phosphate buffered saline
MUC1 MUCIN 1
NaCl Sodium chloride
O/N overnight
OVA Ovalbumin
OxMan Oxidised Mannan
PBS Phosphate buffered saline
PEI Polyethylenimine
PI Propidium iodide
PLL Poly-L-Lysine
RedMan Reduced Mannan
RT Room temperature
SE Standard Error

### EXAMPLE 1

### Production of Reagents, DNA and DNA-Carrier Conjugates

Materials and Methods

Preparations of OxMan-PLL and RedMan-PLL

To oxidised mannan, 14 mg of mannan (Sigma) was dissolved in 1 ml of pH 6 sodium phosphate buffer, followed by the addition of 100 μl 0.1 M sodium periodate (dissolved in pH 6 phosphate buffer) and incubated on ice for 1 hour in the dark. 10 μL ethanediol was added to the mixture and incubated for a further 30 mins on ice. Size exclusion chromatography was used to rid the mixture of sodium periodate and ethandiol and to exchange the buffer. The oxidised mannan mixture was then passed through a PD-10 column (Pharmacia), previously equilibrated with phosphate buffer of pH 8, and the first 2 ml of oxidised mannan eluted using the same buffer used to equilibrate the columns. To the eluted oxidised mannan (2 ml), 1 mg of poly-L-lysine (PLL) (Sigma) dissolved in mouse phosphate buffer solution (mPBS) was then added. The oxidised mannan-PLL (OxMan-PLL) mixture was subsequently left to incubate in the dark at room temperature (RT) over-night (O/N). Finally, the mixture was dialysed with 5 mM NaCl solution (pH8, 0.01 M phosphate) using a membrane that has an molecular weight cut off of 8000 kDa, for 24 h.

Reduced mannan-PLL (RedMan-PLL) was prepared by adding 1 mg sodium borohydride into the OxMan-PLL mixture for 3 h at RT before dialysis. The final concentration of PLL in both OxMan-PLL and RedMan-PLL was 0.5 mg/ml.

Preparation of Mannose-PLL

Preparation of Mannose-PLL was Carried Out with Minor Adjustment to a Previously described method (5). 10 mg of PLL was dissolved in 1 ml of 1 M sodium bicarbonate buffer pH 9.0. 425 μg α-D-mannopyranosylphenyl isothiocyanate dissolved in DMSO, was added to the PLL mixture while vortexing. The mannose-PLL mixture was passed through a PD-10 column, which had been previously equilibrated with 5 mM NaCl solution, and eluted with 2 ml of the 5 mM NaCl solution.

DNA Plasmid Preparation

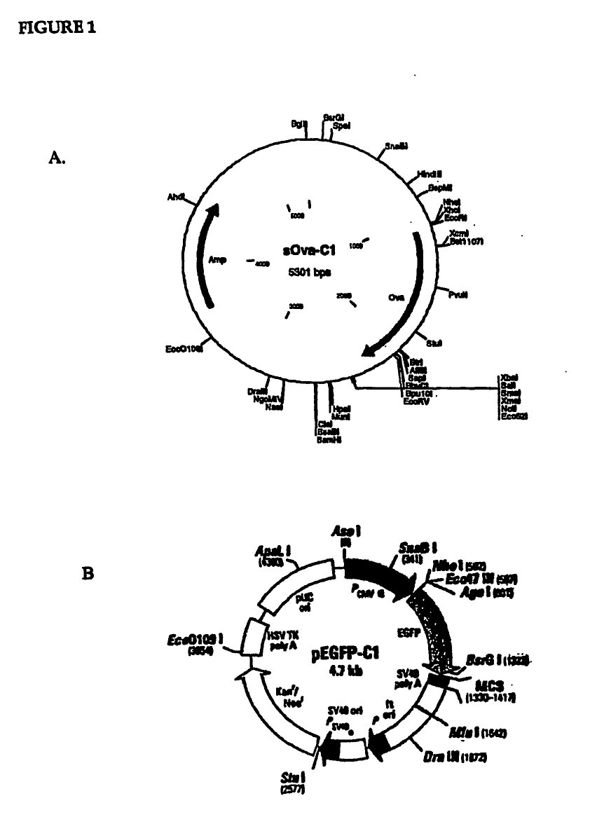

Plasmids used in this example include pEGFP-C1 (for in-vitro studies) and sOVA-C1 (for in-vivo studies) (FIG. 1). pEGFP is a plasmid containing DNA encoding enhanced green fluorescence protein DNA and sOVA-C1 is whole ovalbumin DNA. Plasmids were purified using Qiagen Plasmid Maxi Kit according to the manufacturer's instructions with the exception that bacteria were grown in 2×YT liquid broth instead of LB broth to increase plasmid yield. DNA obtained from the preparation was left to dissolve in distilled water at 4° c. O/N. The concentration of the DNA was determined by its absorbance at 260 nm on a spectrophotometer.

DNA Plasmid Digestion

The DNA was linearised by digestion with the restriction enzyme EcoR1. The amount of EcoR1 (20 units/μL) added was 5% of total weight of DNA yield, with the limitation of the volume of enzyme used being 10% or less than the total volume of the mixture. An equal amount of EcoR1 buffer was also added. The digestion mixture was left to incubate at 37° c. O/N. To confirm the digestion of the DNA, 200 ng of digested plasmid DNA together with an undigested sample and a lambda marker was analysed by gel electrophoresis using 1% agarose gel.

Conjugation Procedure of Carrier-PLL Conjugates and Plasmid DNA

Both OxMan-PLL and RedMan-PLL were complexed to plasmid DNA using the same method. That is, plasmid DNA of various amounts (μg) were dissolved in solution with a final NaCl concentration of 700 mM. To this mixture (plasmid DNA), an equal volume of carrier mixture containing 150 μg of oxidised mannan in a final NaCl concentration of 700 mM was added in a stepwise fashion (10 μl per addition) over 1-2 h. The conjugates, OxMan-PLL-DNA and RedMan-PLL-DNA, were incubated at RT for 30 mins before in vitro assays (using pEGFP-C1 DNA) or prior to injecting into mice (using sOVA-C1).

Mannose-PLL was complexed to plasmid DNA in a different manner to that previously described (6). In the method used, the mannose-PLL was complexed according to the molar charge ratio of PLL:DNA (positive charge of lysine in PLL and negative charge of phosphate present in DNA). In all of the preparations, a PLL:DNA charge ratio (NH₄⁺:PO₃⁻) of 0.75 was used, hence the amount of mannose-PLL used depended on the amount of DNA. Mannose-PLL was added to DNA drop-wise while vortexing, and both preparations (mannose-PLL and DNA) were in a solution of 900 mM NaCl.

Conjugation Efficiency: DNA Precipitation Assay

OxMan-PLL-OVA (DNA) and Mannose-PLL-OVA (DNA) conjugates of various PLL:DNA charge ratio and NaCl concentration were prepared in a similar method to that described above. Conjugates were incubated at RT for 30 mins before centrifugation at 13000 rpm. Supernatant from each conjugate was analysed for DNA content by a spectrophotometer.

Conjugation Efficiency: 0.6% and 1% Agarose Gel Electrophoresis of OxMan-PLL-DNA Conjugates

OxMan-PLL of various amounts (PLL:DNA charge ratio of 0, 0.25, 0.75 and 1) were complexed to OVA DNA at different NaCl concentration (0, 0.9, 1 and 1.1M) (as described above) and incubated at RT for 30 mins before 200 ng of DNA from each preparation was loaded into a 1% agarose gel and run at 100 mV for 1 h. Thereafter, the gel was viewed and analysed under UV.

To further evaluate the complexation efficiency between OxMan-PLL and OVA DNA, the conjugates were analysed on a 0.6% agarose gel electrophoresis. In particular, OxMan-PLL-DNA conjugates formed at varying PLL:DNA molar ratios (0, 0.1, 0.25, 0.4, 0.5, 0.75, 1, 2, 3, 4, 5 and 10) and at 0.7M NaCl were analysed on a 0.6% agarose gel electrophoresis for 1 hour at 100V.

Thereafter, OxMan-PLL-DNA of PLL:DNA ratios r=0.4 and 2 formed at NaCl concentrations of 0, 0.3, 0.7, 1, 2 and 3M were analysed to determine the effect of salt concentration on complexation efficiency.

Results

Conjugation of Mannan (Oxidised and Reduced) to PLL

For oxidised or reduced mannan to act as a carrier for DNA, they must first be conjugated to PLL (or other suitable polycation linker), which acts as a linker between the carrier and DNA via electrostatic interaction (ie the DNA is negatively charged and the PLL is positively charged). In previous experiments using OxMan and RedMan conjugates with peptides or proteins, the conjugation was carried out using 0.1M carbonate buffer pH 9.0 (7). When this buffer was used to conjugate OxMan/RedMan to PLL, precipitation was noted, hence attempts were made to optimise conjugation conditions. Conjugation at different pH (pH 6, 7, 8 and 9) and buffers (phosphate and carbonate) were tested. Using phosphate buffer at pH 8, no precipitation occurred. Therefore, in all subsequent experiments, the conjugation of OxMan to PLL was performed using 0.1 M phosphate buffer at pH 8. The RedMan-PLL conjugate was made by reducing OxMan-PLL with sodium borohydride as described previously (7). Also, in addition to passing the OxMan and RedMan through a size exclusion gel column to remove impurities (which was part of the purifying step in peptide/mannan conjugation in previous studies), at the end of the conjugation steps, OxMan-PLL and RedMan-PLL were dialysed against 0.01 M phosphate buffer with 5 mM NaCl, prior to complexation with DNA.

Efficiency of DNA Binding to OxMan-PLL and Mannose PLL Analyzed by DNA Precipitation Assays

DNA precipitation assays were performed with OxMan-PLL-OVA (DNA) and mannose-PLL-OVA (DNA) conjugates. This previously described assay (8) was used to assess the amount of DNA precipitation by PLL at various salt concentrations. According to the authors, DNA complexed to PLL (bound DNA) would condense and form ordered structures such as spheroids, toroids and rods, which were able to be removed from solution by centrifugation.

For mannose-PLL, the percentage of DNA precipitation increases with increasing PLL:DNA charge ratio. 0 M and 0.9 M NaCl concentration had the highest percentage of DNA precipitation followed by 1 M and 1.1 M. Hence, subsequent preparations of mannose-PLL-DNA were performed at 0.9 M NaCl.

For OxMan-PLL, 50% DNA precipitation was detected for 0 M at PLL:DNA charge ratio of 1. DNA precipitation did not occur in OxMan-PLL at 1 M of NaCl. This pattern of precipitation observed in OxMan-PLL was different to mannose-PLL, hence suggesting that the mechanism involved in complexation of DNA to OxMan-PLL was different from mannose-PLL.

Efficiency of DNA Complexation Analysed by 0.6% and 1% Agarose Gel Electrophoresis of OxMan-PLL/DNA Conjugates

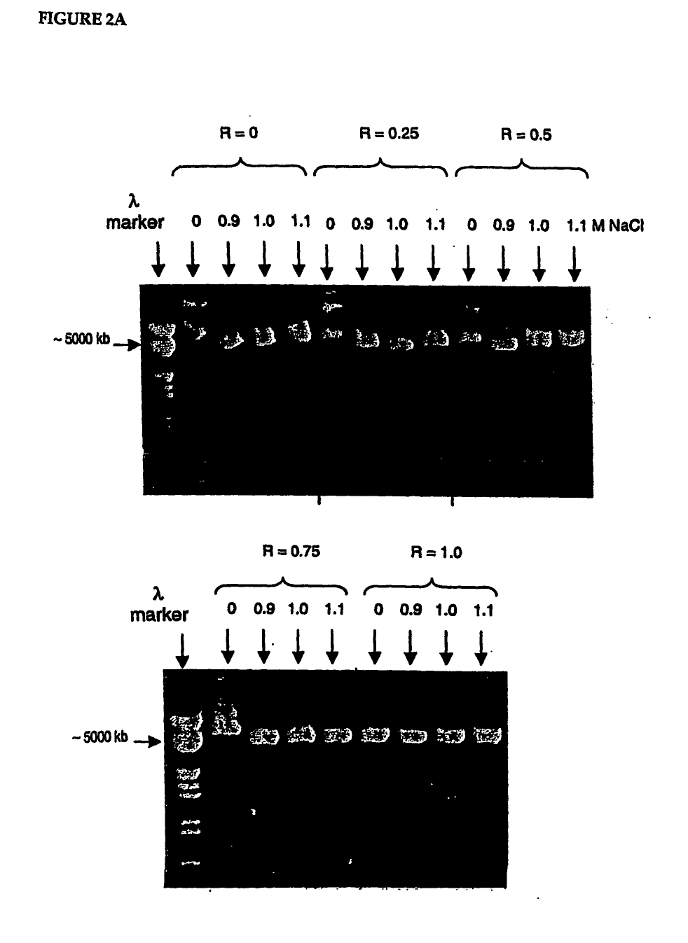

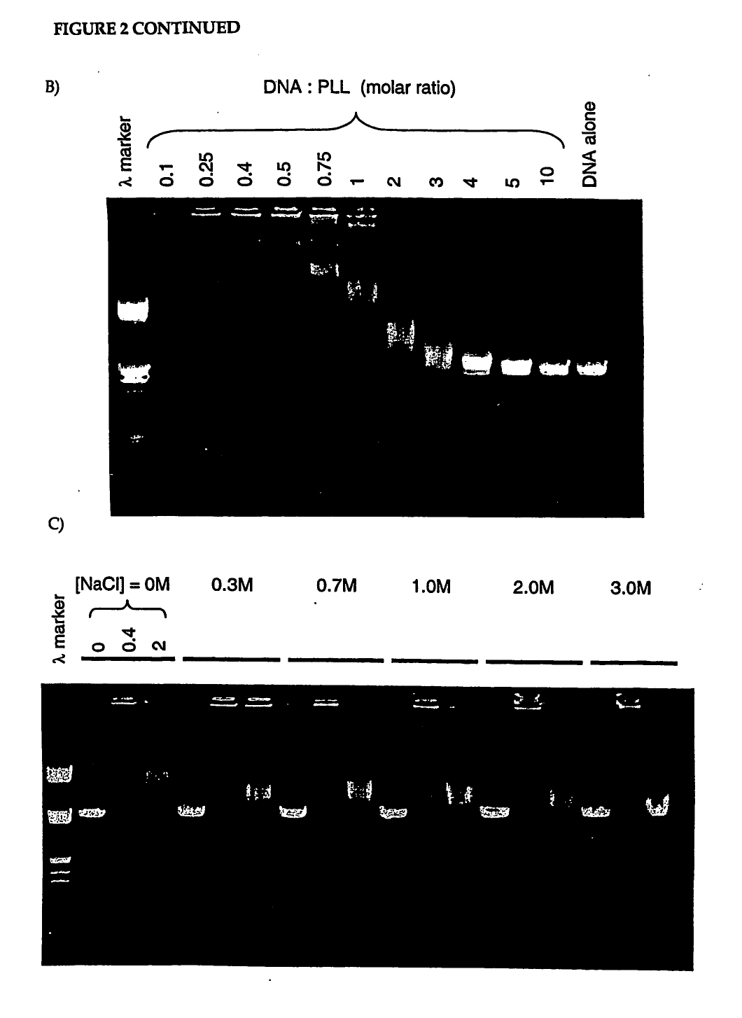

The complexation of DNA to OxMan-PLL was assessed by 1% agarose gel electrophoresis at various NaCl concentrations (0, 0.9, 1 and 1.1M) and PLL:DNA charge ratio (r=0, 0.25, 0.5, 0.75 and 1). Bands seen with 0 M NaCl concentration were different from that of 0.9, 1 and 1.1 M at all PLL:DNA charge ratio (FIG. 2(A)). Hence, there was obvious retardation of DNA in 0 M of NaCl indicating DNA complexation of approximately 50%. This result correlates to the previous data described above in the DNA precipitation assay, where at 0 M, 50% DNA precipitation was observed.

Based on the this experiments, 0.7 M NaCl was chosen to be used for DNA complexation to OxMan-PLL (and RedMan-PLL), not because of the ability to precipitate DNA but because this amount of NaCl would allow optimal electrostatic interactions to occur.

The optimal conditions for complexation of DNA to OxMan-PLL or RedMan-PLL can be determined by varying the ratios of PLL:DNA and varying the NaCl concentration. Optimal proportions of PLL:DNA and salt concentrations vary depending on the plasmids used.

For instance, when further analysis of the complexation efficiency between oxidised mannan-PLL and OVA DNA was conducted (FIG. 2 (B)), it was seen that with DNA alone and PLL:DNA ratio of r=10, a single band of −5 kb was reflected on the gel, but as the ratio was decreased, the level of band retardation decreased proportionally. At PLL:DNA ratio of r=0.1, the DNA was completely retarded and did not migrate away from the well. The binding of the negatively charged DNA to the positively charged PLL of OxMan-PLL therefore appears to result in the DNA losing its charge and hence its capability of migrating to the anode. Accordingly, the level of DNA retardation associated with the varying PLL:DNA ratio value directly relates to different degrees of complexation.

However, when OxMan-PLL-DNA of PLL:PLL ratio of r=0.4 and 2 formed at [NaCl]=0M, 0.3M, 0.7M, 1.0M, 2.0M and 3.0M was analysed on a 0.6% agarose gel electrophoresis for 1 hour at 100V (FIG. 2 (C)), it was seen that that salt concentration does not affect the complexation efficiency at these PLL:DNA ratios.

### EXAMPLE 2

### In Vitro Studies

Materials and Methods

Generation of Mature Murine Bone-Marrow Derived Dendritic Cells (DC)

1.2×10⁷ bone marrow cells from C57BL/6 female mice were cultured on a petri-dish in complete media with the addition of 1000 units/ml granulocyte and macrophage colony stimulating factor (GM-CSF) and 10 ng/ml of interleukin-4 (IL-4). After 6 days of culture, bone marrow cells had the characteristics of mature DCs (high CD11c, CD80, CD86 and MHC class II) capable of stimulating T cells.

Generation of Activated Murine Peritoneal Macrophages

1 ml of 3% Brewer thioglycollate medium (Sigma) was injected into the peritoneal cavity of C57BL/6 female mice. Inflammatory response was allowed for 4 days before the mice were sacrificed, 10 ml of filtered mPBS was flushed and withdrawn from the peritoneal cavity using a 26 gauge 10 ml syringe. Macrophages for use in in-vitro assays were collected from the peritonium, washed, resuspended in complete media, and allowed to adhere in culture wells. Adherent cells were used in all macrophage experiments.

Preparation of Oxidised Mannan-FITC, Reduced Mannan-FITC and Mannose-PLL-FITC

Methods of making oxidised and reduced mannan-FITC were as follows.

To generate oxidised mannan-FITC, 164 μg of 1 mg/ml FITC (dissolved in DMSO) was added to 2 ml of oxidised mannan (7 mg/ml) and incubated O/N in the dark at RT before it was passed through a PD-10 column to separate oxidised mannan from FITC. Reduced mannan-FITC required a 3 h incubation at RT of the oxidised mannan-FITC with 1 mg of sodium borohydride before being passed through a PD-10 column. To make mannose-PLL-FITC, 71 μg of FITC (dissolved in DMSO) was incubated with 1 mg of mannose-PLL at RT O/N before passing the mixture through a PD-10 column.

Flow Cytometry Analysis of DC and Macrophage Cultures

CD11c-PE (monocyte-DC marker), CD3-Cy5 (T cell marker), B220-FITC (B cell marker) and CD14-FITC (monocyte-macrophage marker), PE or FITC isotype control antibodies (all at 1/200 dilutions) were added (in 0.5% BSA/mPBS; 200 μl) to 2×10⁵ DC or macrophages, and incubated for 45 mins at 4° c. After washing (3 times with 0.5% BSA/PBS), the cells were resuspended in 1 μg/ml propidium iodide (PI)/mPBS and immediately analysed using a FACScan flow cytometer. PI was used to gate out dead cells.

Flow Cytometry

The resulting dot plots were interpreted using the Cell Quest Pro software (Becton Dickinson and Company, New York, USA). Analyses were performed on propidium iodide negative cells.

Uptake of Oxidised Mannan-FITC, Reduced Mannan-FITC and Mannose-PLL-FTC by DCs and Macrophages by Flow Cytometry

5×10⁵ mature DCs and 1×10⁶ activated macrophages in a volume of 500 μl and 1 ml of complete media respectively were seeded into each well of 24 well plate. DCs and macrophages were incubated with various amounts (150, 50, 15, 3 and 1 μg) of mannan-FITC, reduced mannan-FITC and mannose-PLL-FITC and incubated at different times (5 mins-3 h). Cells were then collected, washed 3 times in 0.5% BSA/PBS, resuspended in 1 μg/ml Propidium Iodide/mPBs (PI/mPBS) and analysed by flow cytometry.

Expression of eGFP by DCs and Macrophages Incubated with OxMan-PLL-eGFP, RedMan-PLL-eGFP and Mannose-PLL-eGFP Conjugates

5×10⁵ mature DCs and activated macrophages in a volume of 500 μl complete media were seeded into each well of 24 well plate. Conjugates of various composition were added into each well and incubated for 20, 44 and 68 h before they were collected, washed and resuspended in 1 μg/ml PI/mPBS and read in a flow cytometer. FuGENE transfection reagent was used according to the manufacturer's instructions and used as a control. Briefly, 3 μl of FuGENE reagent and 1.5 μl of eGFP DNA was diluted with 95.5 μl of plain media before adding into test wells.

Fluorescence Microscopy of Oxidised Mannan-FITC and Reduced Mannan-FITC Uptake by Macrophages

2.5×10⁵ macrophages in 0.5 ml complete media were seeded into each chamber of a 8 chamber glass slide and allowed to adhere O/N at 37° c. Various amounts (150, 100, 50 μg) of oxidised mannan-FITC or reduced mannan-FITC were added into each chamber and incubated at 37° c. for 2 h. Streptavidin-FITC or Sheep anti-mouse (Fab)₂-FITC were used as negative controls (Silenus). Thereafter, the medium was discarded and each well washed by adding 0.5 ml of mPBS. The chambers were separated from the slides and cover-slips mounted. Slides were then analysed using a fluorescence microscope.

Flow Cytometry: Measurement of Dead Cells by PI Staining

After harvesting the DCs, which had been incubated with OxMan-PLL-DNA, RedMan-PLL-DNA, Mannose-PLL-DNA, mannose-PEI-DNA, PLL-DNA, PEI-DNA, DNA alone, FuGENE and media alone (negative control), they were resuspended in mPBS/PI (1 μg/ml) and analysed by flow cytometry. Dead cells take up PI and the amount (%) dead cells were determined; NOTE: in all flow cytometry analyses, PI positive cells were gated out and analysis done on the PI negative cells.

Inhibition of DNA Synthesis Assay

1×10⁵ DCs were seeded into 96 well U bottom plates in 200 μl volume. Duplicate wells were set up for each condition, which included OxMan-PLL, RedMan-PLL, mannose-PLL, mannose-PEI, PLL, PEI, or nothing. Ranging from 200 μg/ml-1.5 μg/ml. 1 μCi of tritiated thymidine [³H] was added to each well and plates incubated for 20-24 h. Cells were harvested using a cell harvester and [³H] uptake was determined by counting on a Beta-counter.

Results

To evaluate the binding/uptake of various carriers (OxMan, RedMan and mannose linked to FITC) by antigen presenting cells, in-vitro studies were conducted with female C57BL/6 mouse bone marrow derived DCs and activated peritoneal macrophages. The expression of DNA (eGFP) bound to carriers (OxMan, RedMan and mannose-PLL) was also assessed by in vitro grown DCs and peritoneal macrophages.

Fluorescence Microscopy: Binding/Uptake of FITC Labeled OxMan and RedMan by Macrophages

To visualise the actual binding/uptake of oxidised mannan and reduced mannan to the mannose receptor of macrophages, various doses (150, 100 and 50 μg) of oxidised mannan and reduced mannan conjugated to FITC were incubated with macrophages and assessed using a fluorescence microscope. Since FITC alone would readily react with proteins of macrophages and in order to demonstrate that OxMan-FITC and RedMan-FITC specifically bound to macrophages, streptavidin-FITC and sheep anti-mouse (Fab)₂ labeled FITC were used as controls.

Fluorescence was noted by macrophages incubated with OxMan-FITC and RedMan-FITC at all concentrations. Under higher magnification, distinct cell outlines could be seen as well as fluorescence taken up into cells. In both controls, very little staining could be seen. Hence, oxidised and reduced mannan efficiently binds to macrophages. In these experiments, binding/uptake of OxMan/RedMan-FITC by DCs were unable to be performed as they do not adhere to glass slides. Consequently, the uptake/binding of OxMan, RedMan and mannose conjugates by DCs were analysed by flow cytometry.

FACs Profile of DC and Macrophages and Gates Used for Analysis

Flow cytometry was used to characterise the population and profile of DCs and macrophages used for in vitro assays. DC cultures were stained with CD11c-PE (monocyte-DC marker), CD3-Cy5 (T cell marker), B220-FITC (B cell marker) and CD14-FITC (monocyte-macrophage marker) directly labelled antibodies. PE and FITC isotype controls were used as negative controls. Prior to analysis, cells were resuspended in PI (1 μg/ml) to determine and gate out dead cells.

In the FACs profile of DCs stained with PI, there were three distinct populations of cells: gates R1, R2 and R3. Cells in gate R1 stained positive for CD11c (26%), CD3 (24%), B220 (19%) and CD14 (2.8%), PE and FITC controls were negative. The majority of the cells (87.5%) in gate R2 stained very strongly for CD11c (87.5%); other weakly stained cells were CD3, B220 and CD14. These results indicate that a large proportion of R2 cells are CD11c+ (DCs), which also expresses CD40, CD86 and MHC class II molecules. Staining of DCs with both CD11c-PE and OxMan-FITC indicated that a large proportion of DCs (67%) were double positive, thus expression and binding of OxMan to the mannose receptor as previously reported (7).

FACs profile for thioglycollate activated peritoneal macrophages cultures (cells after O/N adherence) do not have cells in gate R1 (as seen in DCs culture) and all the cells are in R2 and R3. Cells in gate R2 stained very strongly with CD11c-PE (95%) and CD14FITC (94%); indicative of macrophage population. Other small cell contaminants, B220+ and CD3+, were also present. Staining of macrophages with both CD11c-PE and OxMan-FITC were 50% double positive (indicative of mannose receptor expression). In all profiles, gate R3 indicated dead cells and were excluded in all analyses. Based on these profiles, R2 was used to analyse the binding/uptake by DC and macrophages in all subsequent experiments.

Kinetics of Binding/Uptake of Carriers by DCs and Macrophages

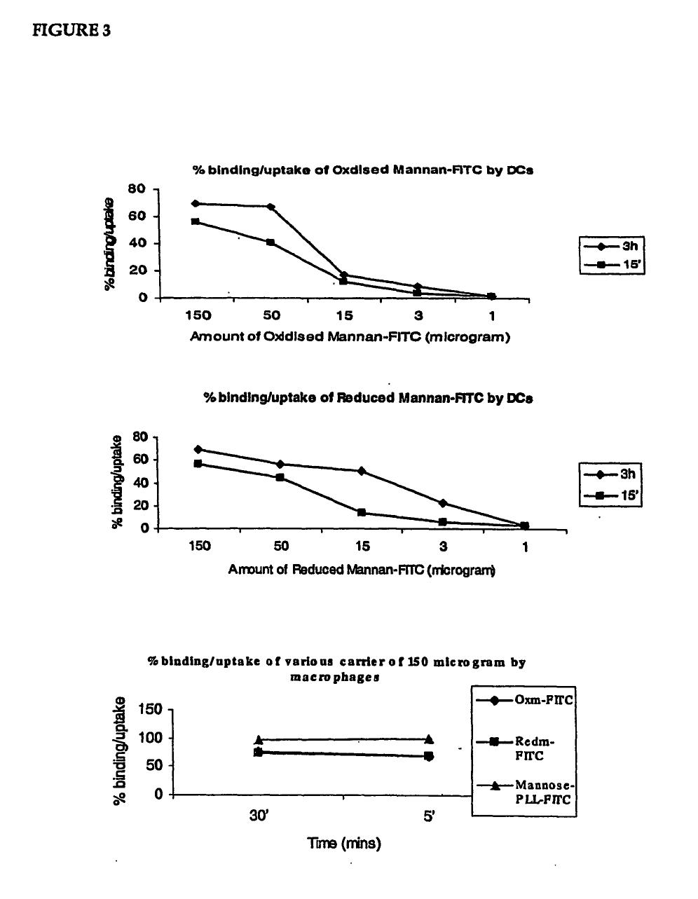

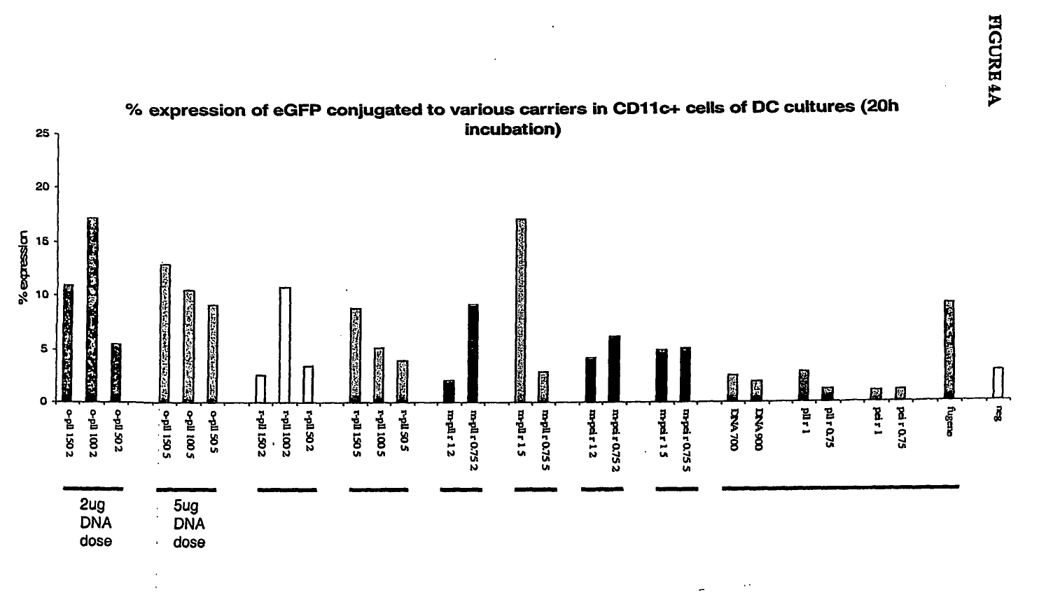

The level of binding/uptake of oxidised mannan, reduced mannan and mannose-PLL by DCs and macrophages was assessed by incubating with FITC coupled carriers of various dose and incubation times. DCs incubated with decreasing doses (150, 50, 15, 3 and 1 μg) of OxMan-FITC or RedMan-FITC titrated for both 3 h and 15 minute incubation times (FIG. 3). 150 and 50 μg of either OxMan-FITC and RedMan-FITC gave optimal uptake/binding, which was rapidly decreased at 15 and 3 μg dose. These results demonstrate that both OxMan and RedMan bind efficiently to DCs (binding to the mannose receptor) and a dose dependent binding is observed. Mannose-PLL-FITC was also incubated with DCs but majority of the cells (90-100%) were stained positive for PI and were not included in the analysis (see below for toxicity effects of mannose-PLL).

The percentage of uptake of OxMan-FITC and RedMan-FITC by macrophages was similar to DCs; Mannose-PLL-FITC (the proportion of cells that stained negative for PI) stained higher. The efficiency of binding/uptake seen by all carriers is similar, and no difference is seen in percentage uptake between 30 mins and 5 mins. Therefore, similar to DCs, mannan readily binds and/or is taken up by macrophages. In FIG. 3, uptake of 150 μg mannan/mannose-FITC by macrophages at 3 h and 15 mins (DCs) and 30 mins and 5 mins (macrophages) are presented. The above data suggests that OxMan, RedMan and mannose-PLL binds and/or is taken up efficiently by both DCs and macrophages.

Expression of eGFP DNA by DCs and Macrophages

The ability of DCs and macrophages to express genetic materials complexed to the carriers was evaluated. eGFP DNA was used to complex with the various carriers and incubated with DCs for 20 h, 44 h and 68 h. Most studies investigating expression of exogenous DNA in transfected cells are usually analysed between 20 to 44 h of incubation, hence 20 h was chosen as the first time point of analysis in this study. Mannose-PEI, like mannose-PLL, is a widely used DNA delivery vehicle used to target macrophages and was included in this experiment to compare immune responses generated with different linkers. FuGENE is a commercially available transfection reagent kit that transfects foreign DNA into cells via lipofection and was included in the study as an alternative method of transfection. As controls, DNA alone, PLL-DNA and PEI-DNA were also tested. In addition, to determine if the NaCl concentration in the solution of DNA affects the level of transfection, DNA alone in 700 mM and 900 mM NaCl solution were tested.

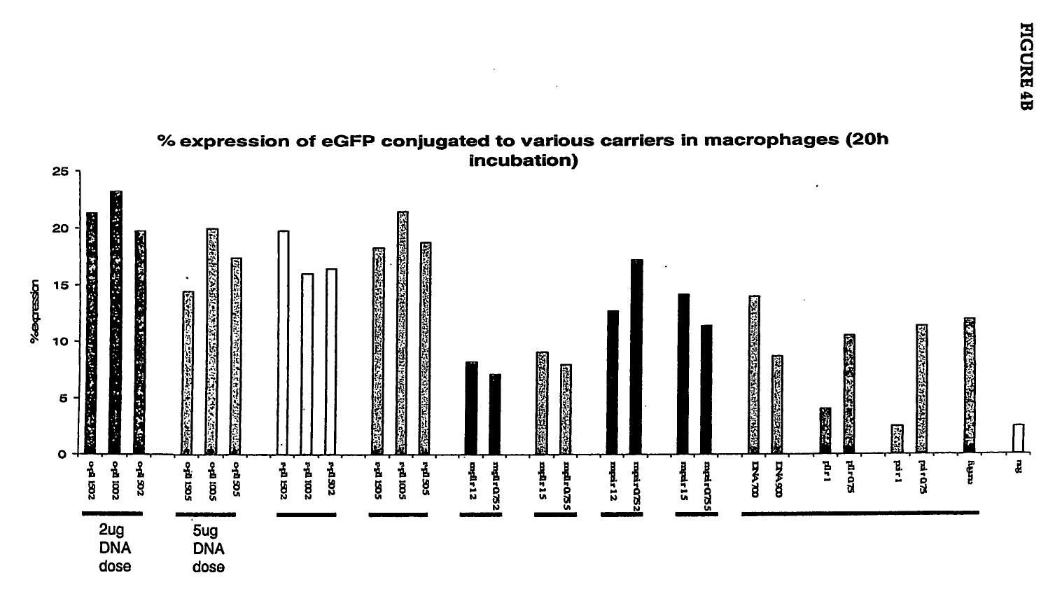

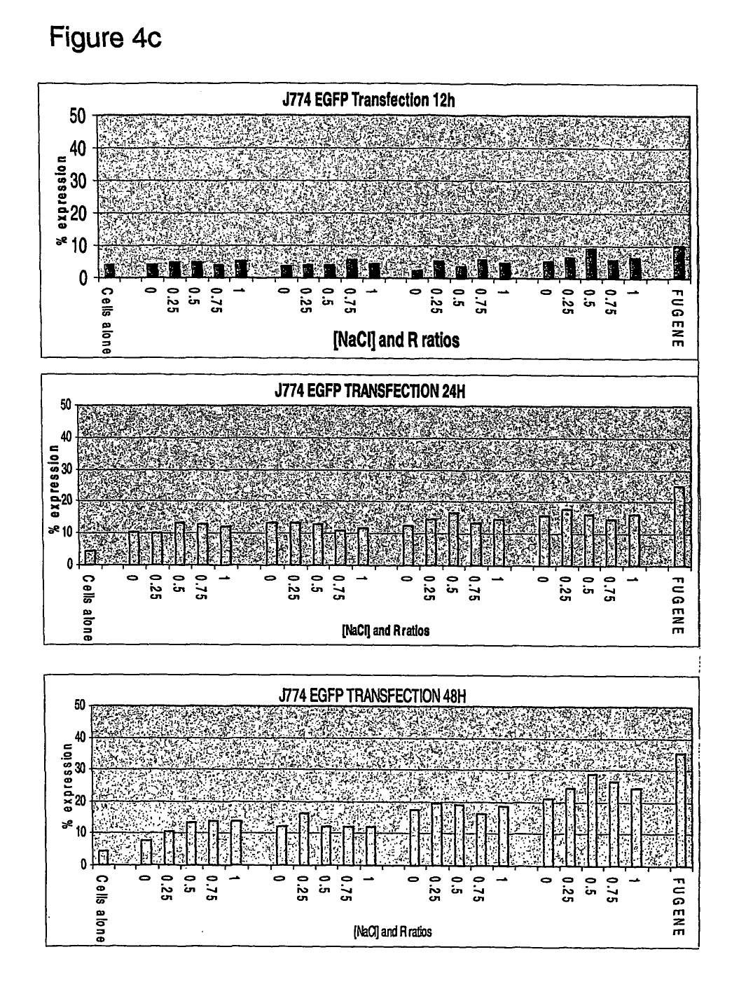

DC cultures were analysed for expression of fluorescence using gate R2 (as defined previously). eGFP was expressed for 20 h and differential expression of fluorescence was detected when complexed to different carriers (FIG. 4). Expression levels were not affected by the salt concentration of the solution. 100 μg OxMan-PLL with 2 μg of DNA had the highest level of expression and overall, OxMan-PLL groups had higher expression than FuGENE and other carrier groups. OxMan-PLL and RedMan-PLL showed the same pattern of expression when complexed to 2 μg and 5 μg of DNA (FIG. 4). At 2 μg DNA, 100 μg of mannan showed the highest expression followed by 150 and 50 μg but at 5 □μg DNA dose, expression decreased as the amount of mannan decreased. Mannose-PLL and mannose-PEI showed lower levels of expression compared to OxMan-PLL and RedMan-PLL except for mannose-PLL/r=1/5 μg DNA. Different PLL:DNA charge ratios in mannose-PEI and mannose-PLL groups showed different expression level. At 2 μg DNA dose for mannose-PLL and mannose-PEI, r=0.75 had better expression level compared to r=1, and vice versa for 5 μg DNA dose (FIG. 4). PLL, PEI and DNA alone groups gave low expression compared to groups with mannose or mannan conjugated, thus showing that receptor-mediated gene transfer resulted in better transfection in DCs.

Based on the findings that the expression of eGFP level peaked at 20 h and 44 h-68 h showed no expression by DC, the same experiment was performed on macrophages for only 20 h. For macrophage cultures (FIG. 4), PI negative cells (alive cells) were analysed. Compared to DC cultures, expression of all groups was much elevated. OxMan-PLL and RedMan-PLL groups showed similar level of expression. Mannose-PEI groups generally gave higher expression than mannose-PLL groups. The level of expression by DNA alone at 2 different salt concentrations was almost as high as those seen in mannose-PLL and mannose-PEI group and, further, 700 mM NaCl concentration gave higher expression compared to 900 mM NaCl.

These experiments demonstrated that macrophages and DCs are different in the kinetics of expression of DNA complexed to different carriers, however OxMan-PLL and RedMan-PLL were shown to be the most efficient receptor-mediated gene transfer ligands taken up and expressed by cells.

Toxicity of PLL/PEI (+/−Conjugates) as Assessed by PI Staining

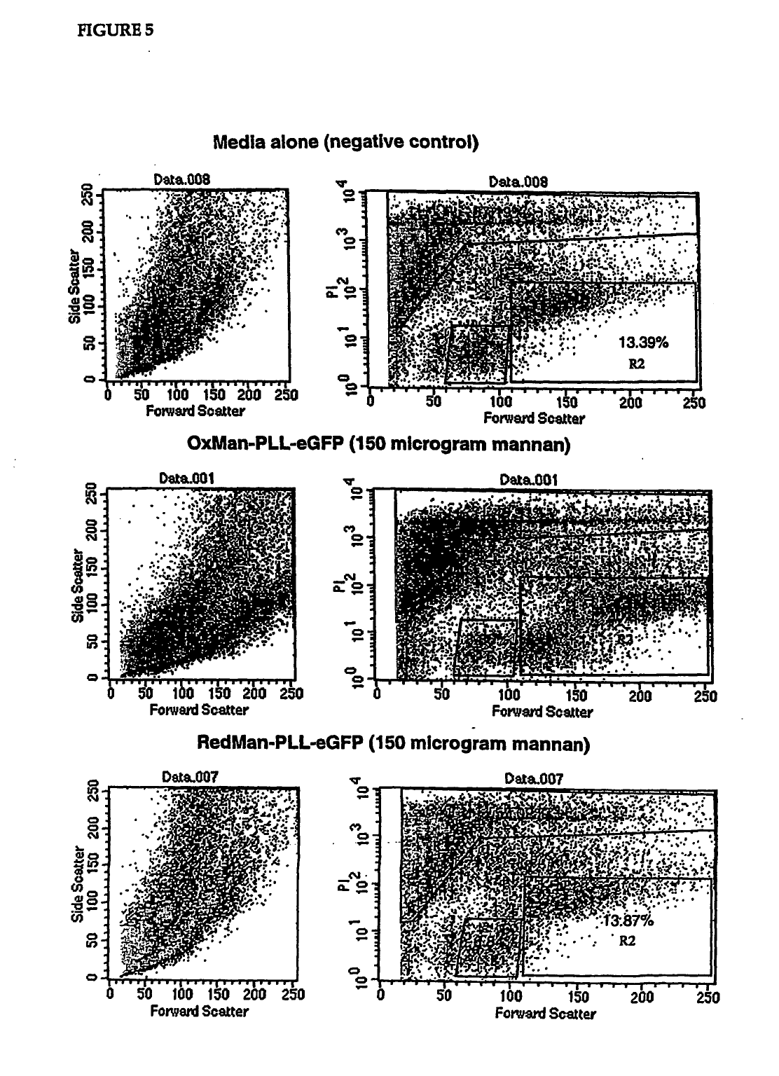

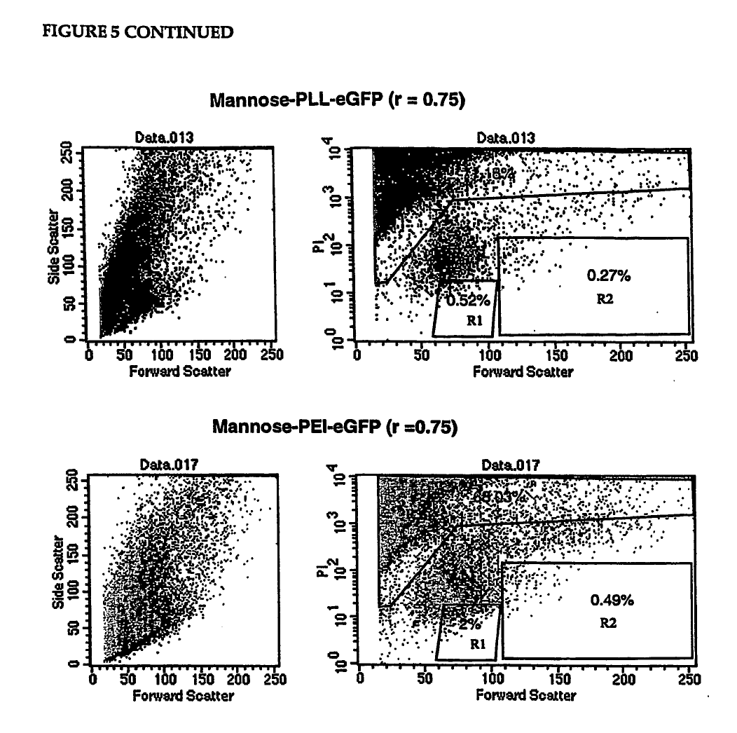

FACs profiles of DC and macrophage cultures constantly showed the same pattern of cell viability/death when incubated with various carriers. Cells incubated with mannose-PLL, mannose-PEL, PLL and PEI as carriers (for eGFP and FITC) consistently yielded a larger percentage of PI positive stained cells compared to cells incubated with DNA alone, OxMan-PLL and RedMan-PLL (FIG. 5). FACs profile of these DC cultures were analysed for the percentage of alive and dead cells present and compared between carriers to understand the level of toxicity each had relative to each other.

DCs incubated with OxMan-PLL-DNA (eGFP), RedMan-PLL-DNA (eGFP), mannose-PLL DNA (eGFP) and mannose-PEI DNA (eGFP) for 20 h were assessed for percentage alive/dead cells by PI staining. The group that had media alone (negative control) had the highest percentage of alive cells (FIG. 5), which was 22.36%. Such a low % of alive cells was expected as the DCs used were from bone marrow cells cultured for 6 days with GM-CSF and IL4. In contrast, non-DC precursors that are not stimulated by the cytokines would not remain viable after 6 days of incubation. Mannose-PEI and PEI is the most toxic to DCs, followed by PLL, mannose-PLL, RedMan-PLL and OxMan-PLL (FIG. 5).

Inhibition of DNA Synthesis Assay

To further analyse the degree of toxicity by each carrier on DCs, DCs were incubated with carriers (OxMan-PLL, RedMan-PLL, mannose-PLL, mannose-PEI, PLL and PEI) at a range of different concentrations. Thymidine was added to these cultures to measure the level of DNA activity/synthesis cells had in the presence of the carriers, as an indication of their viability in culture.

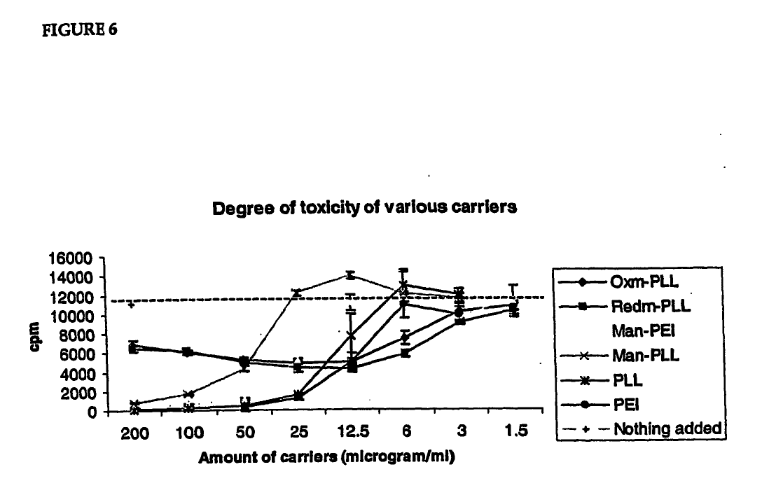

From 200 to 25 μg/ml, mannose-PLL, mannose-PEI, PLL and PEI inhibited most DNA activity of DCs, OxMan-PLL and RedMan-PLL gave the same level of inhibition throughout (FIG. 6). Although at the lower concentration range (12.5 to 1.5), the level of inhibition decreased rapidly in mannose-PLL, mannose-PEI, PLL and PEI, and seemed to be even less toxic than OxMan-PLL and RedMan-PLL. Hence, these results match the pattern of dead cells seen in flow cytometry when DC and macrophages cultures were incubated with various carriers. Mannose-PLL and PLL alone were very toxic to cells; OxMan-PLL and RedMan-PLL appeared to mask the toxic effect of PLL.

Discussion

In vitro experiments were performed to study the interaction between these carriers with DCs and macrophages, followed by expression of protein after carrier-DNA complex uptake.

Results of in vitro studies revealed that OxMan and RedMan have potential for the efficient delivery and expression of genetic materials into DCs and macrophages. The ability for a specific ligand to recognise and have affinity for the receptor is the foremost important step in receptor-mediated gene transfer. This example has demonstrated that OxMan and RedMan are able to bind to and/or be taken up at similar levels by DCs and macrophages using immuno-fluorescence microscopy and by flow cytometry. The binding/uptake efficiency of OxMan and RedMan by DCs and macrophages was efficient and fast (within 5 mins). This example also demonstrated that OxMan and RedMan-PLL complexed with DNA (eGFP) added to DCs and macrophages, were able to bring about expression of eGFP at high levels as compared to DNA alone, mannose-PLL, mannose-PEI, and also FuGENE (control). The enhanced transfection efficiency by OxMan-PLL is believed to be due to the effect of aldehyde groups on the oxidised mannan causing escape of the endocytosed exogenous genetic material before it is transferred to the lysosomes to be degraded by nucleases contained within the lysosomes, thus allowing for the DNA to be brought to the nucleus and be transcribed. The results showed that while the percentage of transfection by RedMan-PLL in DCs was not as high as oxidised mannan, it was still higher than the other groups tested. One possible explanation for this observation is that although it does not have aldehydes present to cause endosomal escape, the presence of mannan, which has a high molecular weight (50000 to 1000000 kDa), could protect PLL-linked DNA in the external environment and in the lysosomes, so that relatively more DNA can still survive (by not being degraded by lysozymes) to be translocated to the nucleus. Further, in the example, complexation of DNA to OxMan-PLL and RedMan-PLL at various mannan concentration, and mannose-PLL and mannose-PEI at various PLL:DNA charge ratio, was demonstrated to have different transfection efficiency, thus having a higher dose of carrier complexed to a fixed amount of DNA does not necessarily mean that the transfection rate is higher. Moreover, the example showed that OxMan-PLL and RedMan-PLL was less toxic than mannose-PLL and mannose-PEI.

### EXAMPLE 3

### In Vivo Studies

Materials and Methods

Mice and Immunisations

6 to 10 weeks old in-bred female C57BL/6 mice were used in all experiments. Intra-dermal injections were performed by injecting 50 μL of various DNA construct mixtures into the base of the tail of each mouse. Mice were immunised 2-3 times every 2 weeks. 10-14 days after the last immunisation mice were culled by CO₂ asphyxiation and immune responses were assessed by observing either the absence or extent of tumour growth.

Preparation of medium Used in Cell Cultures and Assays

RPMI 1640 medium was used in cell cultures for all experiments conducted in this study. Complete media was supplemented with 10% heat inactivated foetal calf serum (FCS), 4 mM L-glutamine, 100 units/ml penicillin, 100 mg/ml streptomycin sulphate, 100 mM β-mercaptoethanol and 10 mM HEPES. Supplemented and non-supplemented media are termed complete and plain media respectively in this report.

Preparation of Cells for Use in ELISPOT and Proliferation Assays

Splenocytes to be added to the wells of ELISPOT or proliferation assay plates were prepared by separating the spleen cells in complete media and then passing through a cell strainer. Red blood cells were lysed in 5 ml ACK lysis buffer (1M potassium hydrogen carbonate, 0.15M ammonium chloride, 0.1 mM EDTA) at 37° c. for 10 mins. Cells were washed twice in plain media and centrifuged at 1500 rpm, 4° c. for 5 mins. Spleen cells were re-suspended in 10 ml complete media and the cells were counted using a haemocytometer.

ELISPOT Assay

Plates (96 well, MAIP plates) were pre-wet with 50 μl of 70% ethanol followed by 6 times washing with 200 μl mPBS in sterile conditions. 70 μl of 5 μg/ml (in mPBS) IFN-γ or IL-4 coating antibodies were added into each well, and incubated 0/N incubation at 4° c. Plates were blocked by adding 200 μl of complete media (supplemented with 10% FCS) and incubated for 2 h at 37° c. The blocking media was discarded and peptide antigens were added into each defined well. Types of peptide antigen and their respective final concentration in each well were: 10 μg/ml of whole ovalbumin (OVA), OVA CD8 epitope (peptide sequence: SIINFEKL (SEQ ID NO: 1)) and OVA CD4 epitope (peptide sequence: ISQAVHAAHAEINEAGR (SEQ ID NO: 2)) and 1 μg/ml of Con A (internal positive control). mPBS was used as a negative control. Triplicate wells were set up for each condition. 5×10⁵ spleen cells in 100 μl complete media were seeded into each well and incubated at for 18 h at 37° c. Plates were incubated for 2 h at RT with anti-murine IFN gamma mAb-biotin or anti-murine IL-4 mAB-biotin respectively. Plates were washed (as above) and streptavidin-ALP was added at 1 μg/ml and incubated for 30 mins at RT. Spots of activity were detected using a colorimetric AP kit and counted using an ELISPOT plate reader. Data are presented as mean spot forming units (sfu) per 0.5 million cells +/− standard error of the mean (SE).

Proliferation Assay

2×10⁵ splenocytes in a volume of 100 μl complete media were seeded into each well of a 96 well round bottom plate and cultured with the same peptide antigens of the same amount used in ELISPOT assays. Cells were cultured for 5 days. Every 24 h for 5 days, 1 μCi of Thymidine [³H] was added to the wells and incubated for 6 h. [³H] uptake was measured by harvesting cells into glass fibre filters and radioactive counts were detected by a scintillation counter (β-counter).

ELISA Assay

Serum was collected from eyebleeds of mice (pre-bleeds, and 2 weeks after every injection). Polyvinyl chloride micro-titre plates coated with chicken egg ovalbumin (10 μg/ml in coating buffer {0.05M Sodium bicarbonate, pH 9.6}) O/N at 4° c. Plates were blocked with 2% bovine serum albumin (BSA) PBS for 2 hour at 37° C. in a humidified box. Plates were washed with mPBS 0.05% Tween-20 5 times and incubated for 2 h at RT with mouse sera from immunised mice at various dilutions (1/50, 1/150, 1/450, 1/1350, 1/4050, 1/12150, 1/36450 and 1/109350) then washed 15 times, incubated with horseradish-peroxidase-conjugated sheep anti-mouse Ig for 2 h at RT. After washing the plates, developing buffer (5 ml of ABTS buffer {1M sodium hydrogen phosphate, 1M Citric Acid, pH 4.5}, 100 μl ABTS stock and 4 μl H₂0₂) for 15-30 mins was added. Plates were read on Fluostar Optima microplate reader at 405 nm.

Statistical Analysis

Mean values were compared using the two-tailed Student's t-test. P value threshold of P<0.05 indicates a statistically significant difference.

Results

From the preceding in vitro studies, it was clear that OxMan and RedMan-FITC efficiently bind to DC and macrophages. That is, OxMan/RedMan-PLL-DNA was readily taken up by DC and macrophages and DNA (eGFP) was expressed as a protein within 20 h. Based on these findings, in vivo studies were undertaken.

In particular, ovalbumin (OVA) DNA complexed to mannan or mannose via PLL was used to immunise C57BL/6 mice. OVA DNA was used as a model antigen as the CD8 (SINFEKL (SEQ ID NO: 1)) and CD4 (ISQAVHAAHA AGR (SEQ ID NO: 2)) epitopes from OVA in C57BL/6 mice were known and, therefore, in-vivo studies could be performed to analyse the type of immune response generated. Mice were immunised with either OxMan-PLL-DNA, RedMan-PLL-DNA or mannose-PLL-DNA and immune responses were analysed by proliferation assays (to assess both CD4 and CD8 T cells) and cytokine secretion (IFN-gamma or IL-4) of T cells by ELISPOT assays. CD4 epitope and CD8 epitope peptides are hereinafter referred to as “CD4” and “CD8”.

In the first in-vivo study performed, the immune response generated by OxMan-PLL-DNA, RedMan-PLL complexed with 10 μg of OVA DNA was assessed. DNA alone and PLL-DNA were used as controls. Mice (6 mice per group) were given 3 injections, with 10-14 days apart. 2 weeks after the 2^{nd} and 3^{rd} injections, mice were sacrificed and immune responses evaluated. Serum was collected from each mouse prior to each injection and 2 weeks after the final injection.

Pilot Study—Proliferation Assay

To characterise the T cell response, a proliferation assay was used to detect the level of antigen-specific T cells, by measuring the thymidine uptake of T cells due to proliferation induced in the presence of peptides. The assay was performed on days 3, 4 and 5 after thymidine was added to determine the peak proliferation of the T cells. In all mice, proliferation was the highest on day 3 of the analysis and reduced sharply on day 4 and 5 (FIG. 7). The peak however could be earlier than day 3. The maximum [³H]-thymidine uptake between each group was different to various peptides. Mice injected with RedMan-PLL-DNA generated T cells, which proliferated primarily to CD4 OVA T cell epitope. While mice immunised with OxMan-PLL-DNA generated T cells which recognised only the CD8 epitope. T cells from mice immunised with DNA alone and PLL-DNA, however, did not recognise and proliferate to any of the OVA peptides added. The polyclonal mitogen, concanavalin A (ConA) was used as a positive control and no peptide was used as a negative control. The proliferation assays were done after two immunisations.

Pilot Study—ELISPOT Assay

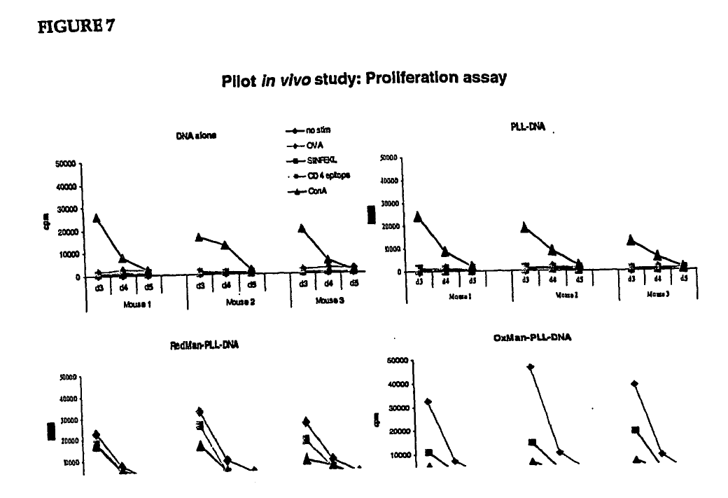

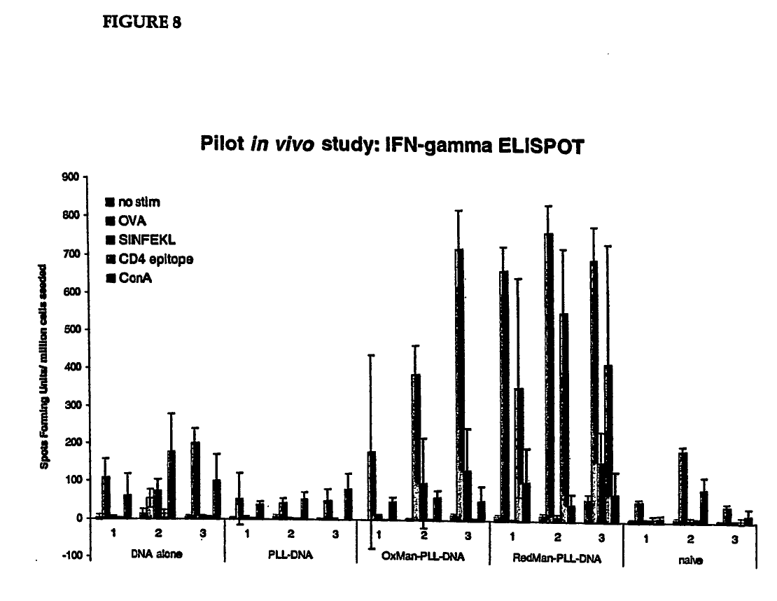

ELISPOT analyses specific T cell responses to antigens by detecting the secretion of specific cytokines. In this experiment, IFN-γ secretion by T cells in mice after 2 injections was analysed. The pattern of stimulation in the ELISPOT assay was similar to the proliferation assay analysis whereby, mice injected with OxMan-PLL-DNA generated IFN-γ secreting T cells in the presence of CD8 peptide and RedMan-PLL-DNA generated IFN-γ secreting T cells in the presence of CD4 peptide (FIG. 8). Mice injected with DNA alone and PLL-DNA induced weak responses to whole ovalbumin but no responses to CD4 or CD8. ConA responses were weak in these experiments and in the proliferation assay. ConA, loses its activity if freeze thawed too many times; this maybe one reason for the observed. In the subsequent experiments, fresh ConA was made and high ConA responses were seen (see below).

Pilot Study—ELISA

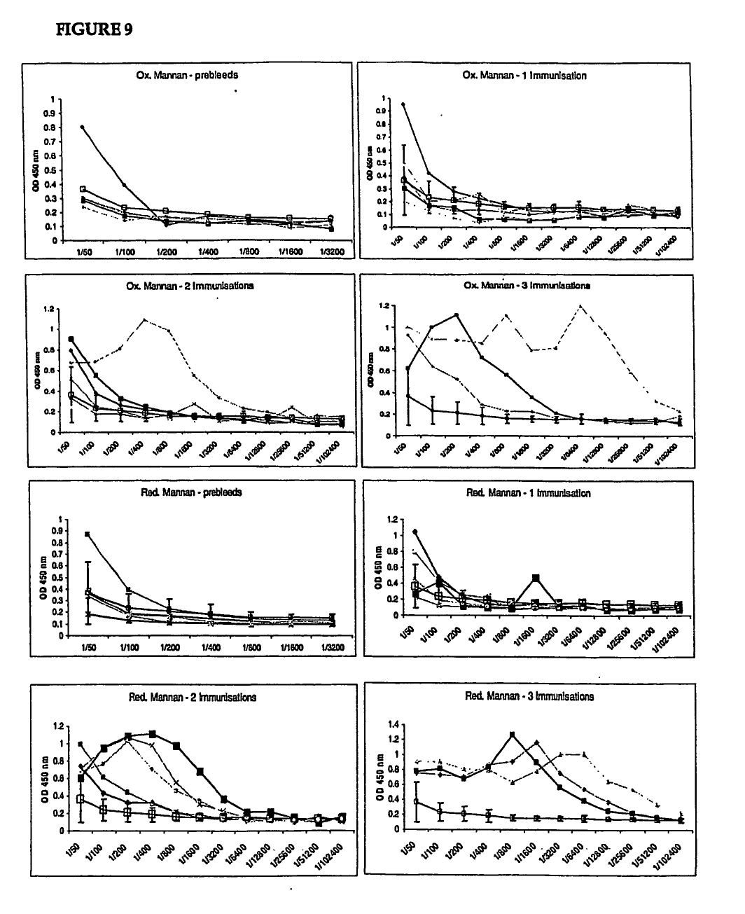

ELISA was used to detect the level of antigen-specific antibodies (total immunoglobulin) present in each mouse, as an indication of humoral immune responses. Serum obtained from eyebleeds of each mouse was collected 2 weeks after each injection. Only mice immunised with OxMan-PLL-DNA or RedMan-PLL-DNA induced high antibody levels, while DNA alone and PLL-DNA did not induce any antibodies (FIG. 9). Antibody levels started to rise after 2 injections (6 mice/group) in the RedMan-PLL-DNA group and 3 injections (3 mice/group) in OxMan-PLL-DNA group and after the 3^{rd} injection, the former induced a higher antibody level than the latter. Thus, RedMan-PLL-DNA generated the strongest antibody responses followed by those generated by OxMan-PLL-DNA.

Extension Studies

Further in-vivo assays were conducted with, in addition to OxMan-PLL-DNA, RedMan-PLL-DNA, PLL-DNA and DNA alone, (1) Mannose-PLL-DNA—mannose-PLL (a widely used receptor mediated gene delivery vector, included for comparison), (2) a 50 μg DNA dose for each carrier, (3) performing proliferation assays and ELISPOT on the pooled spleens of mice within each group, (4) in proliferation assays, assessing results on days 1 to 5 rather than days 3 to 5 so as to determine the proliferation peak by T cells, and (5) in ELISPOT assays, measuring IL-4 in addition to IFN-γ. The experiments were repeated 2 times with 2 to 4 mice per group.

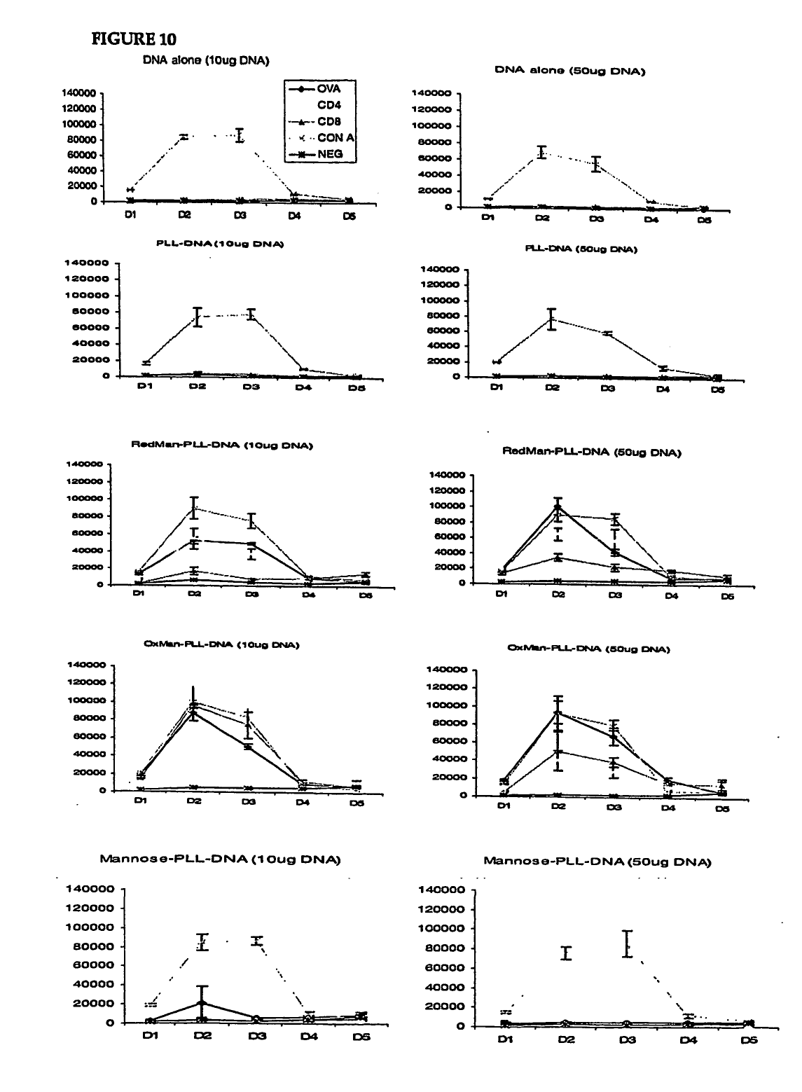

Proliferation assays were performed on days 1 to 5 instead of days 3 to 5. T cell proliferation peaked at day 2 and started to reduce sharply by day 4 (FIG. 10). Like previous results, mice immunised with RedMan-PLL-DNA (10 μg) generated T cells, which proliferated primarily to CD4 OVA T cell epitope. However, at a higher immunisation dose (50 μg), T cells were generated which recognised both CD4 and CD8 OVA T cell epitopes. There is a significant difference (P<0.05) in CD8 responses between 10 and 50□ μg DNA dose of RedMan-PLL on day 2 and 3 of the proliferation assay. Mice immunised with OxMan-PLL-DNA generated T cells which recognised only the CD8 epitope (at lower immunisation dose; 10 g), however at a higher immunisation dose (50 μg) both CD4 and CD8 T cell epitopes were recognised by T cells (CD8 epitope however much lower at higher doses). Significant differences (P<0.05) were detected in CD4 responses between 10 and 50 μ□g DNA dose of OxMan-PLL on day 2 and 3 of the proliferation assay. Overall, the T cell response to whole ovalbumin was much higher than to individual CD4 or CD8 peptides. Mannose-PLL-DNA, PLL-DNA and DNA alone did not induce any proliferative responses to either the CD4 or CD8 epitope peptides.

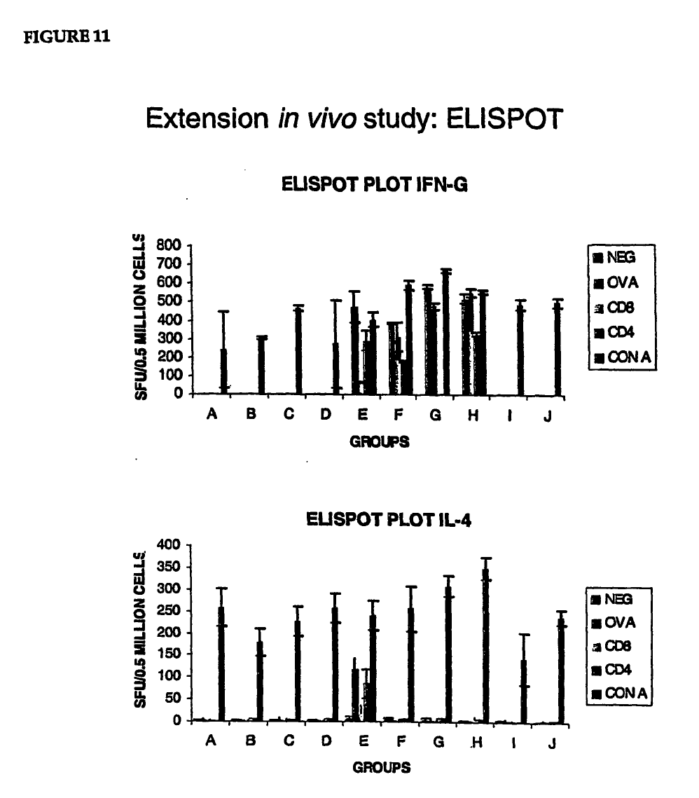

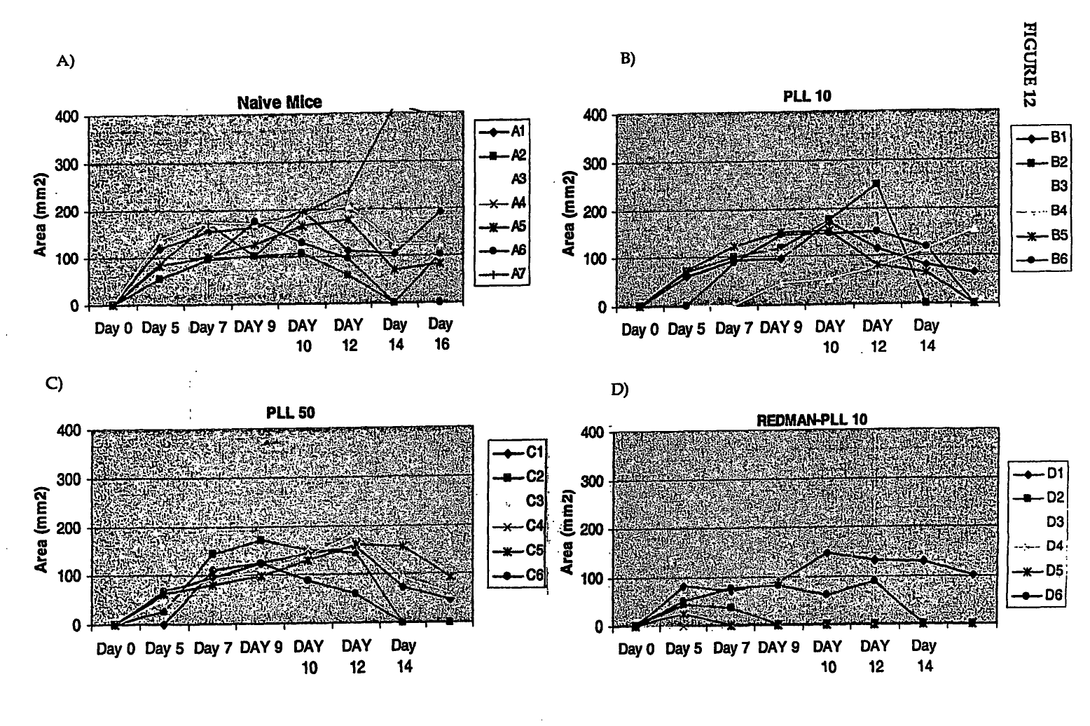

ELISPOT assays were conducted to measure both IFN-γ and IL-4 secretion by T cells. The IFN-γ cytokine secretion demonstrated similar results as to the proliferation assay, whereby, at a low immunisation dose (10 μg) OxMan-PLL-OVA induced primarily CD8 T cell responses and RedMan-PLL-OVA CD4 T cell responses; at higher doses (50 μg), both CD4⁺ and CD8⁺ T cells secreted IFN-γ from both OxMan-PLL-OVA or RedMan-PLL-OVA immunised mice (FIG. 11). Significance differences (P<0.005) in the CD8 response of RedMan-PLL at 10 and 50 μg DNA dose was observed. Generation of IL-4 cytokine by T cells was only demonstrated in the RedMan-PLL-OVA (10 μg dose). In both cytokine secretion assays (IFN-gamma or IL-4), Mannose-PLL-DNA, PLL-DNA and DNA alone did not induce T cells which recognised either the CD4 or CD8 epitope peptides.

In both proliferation and ELISPOT assays, OxMan and RedMan were mixed with DNA (10 and 50 μg) (without PLL linker) and injected into mice. Neither induced T cells and the levels were similar to those of other controls (data not shown). This demonstrates that OxMan or RedMan both need to be linked to DNA and on their own do not induce non-specific immune responses. From the above results, it can be seen that the oxidised/reduced mannan delivery system is very versatile and can be used to target DNA to the mannose receptors. They can both generate strong cellular (CD4 and CD8 T cell) immunity.

Discussion

In vivo experiments were conducted in mice to assess immune responses elicited by injecting with DNA complexed to OxMan and RedMan via PLL as a linker. The results of these experiments were surprising.

In previous studies using protein conjugated to OxMan and RedMan, the OxMan conjugate induced primarily CD8⁺ type immune responses whilst RedMan induced primarily CD4⁺ type immune responses (Lofthouse, S A et al, 1997; Apostolopoulos, V et al, 2000; and Apostolopoulos, V et al, 1995). Herein, at low DNA doses (10 μg), OxMan-PLL-DNA and RedMan-PLL-DNA induced primarily CD8⁺ and CD4⁺ responses respectively (ie as demonstrated in both proliferation assays and IFN-γ ELISPOT assays), but at higher DNA doses (50 μg) both CD8⁺ and CD4⁺ immune responses were induced by OxMan-PLL-DNA and RedMan-PLL-DNA.

A possible mechanism by which OxMan-PLL-DNA, at low DNA dose, induces a primarily CD8⁺ immune response is through the aldehydes acting internally in the cell, on the proteasomes, Golgi apparatus or endoplasmic reticulum, which may enhance MHC class I peptide presentation and stimulation of CD8⁺ T cells. Further, the aldehydes in the oxidised mannan may directly stimulate T cells. At higher doses of DNA, it is possible that more antigenic peptides are produced to get processed as MHC class I/antigen complex and also be secreted to the exterior of the cell. These secreted antigenic peptides may then be phagocytosed by the DCs or macrophages and be processed as an antigen via exogenous pathway to induce CD4⁺ immune responses.

It was surprising that RedMan-PLL-DNA at 10 μg dose induced CD4⁺ immune responses and not CD8⁺ immune responses. At higher doses (50 μg), the immune responses were similar to OxMan-PLL-DNA in that both CD4⁺ and CD8⁺ immune responses were generated. One would presume that immune responses generated to RedMan-PLL-DNA would be similar to OxMan-PLL-DNA because antigen is expressed endogenously. The preferential CD8⁺ immune responses to OxMan-PLL-DNA and CD4⁺ immune responses to RedMan-PLL-DNA were very surprising.

### EXAMPLE 4

### Challenge of Immunised Mice with EG7 Cells

Materials and Methods

Mice and Immunisations

6 to 10 week old in-bred female C57B1/6 mice were used. Intradermal injections were performed by injecting on day 0 and 14, in the base of the tail of each mouse, 50 μl of: (a) PBS; (b) PLL-DNA (10 μg); (c) PLL-DNA (50 μg); (d) RedMan-PLL-DNA (10 μg); (e) RedMan-PLL-DNA (50 μg); (f) OxMan-PLL-DNA (10 μg); and (g) OxMan-PLL-DNA (50 μg). Six mice were injected with each dose. The DNA was prepared from the sOVA-C1 plasmid as described in Example 1.

Challenge with EG7 Cells

On day 28, each mouse was challenged with a subcutaneous dose of 1×10⁷ EG7 cells (OVA-EL4) expressing whole OVA. The EG7 cells grow in C57BL/6 mice to a maximal tumour size and are then rejected due to the immune responses to the foreign OVA antigen. Immune responses to the tumour can therefore be measured by either the tumour failing to grow or through growing the tumour to a lower maximal size than the tumours in control mice treated with PBS. Tumour growth was monitored by measuring the two perpendicular diameters using a caliper.

Results and Discussion

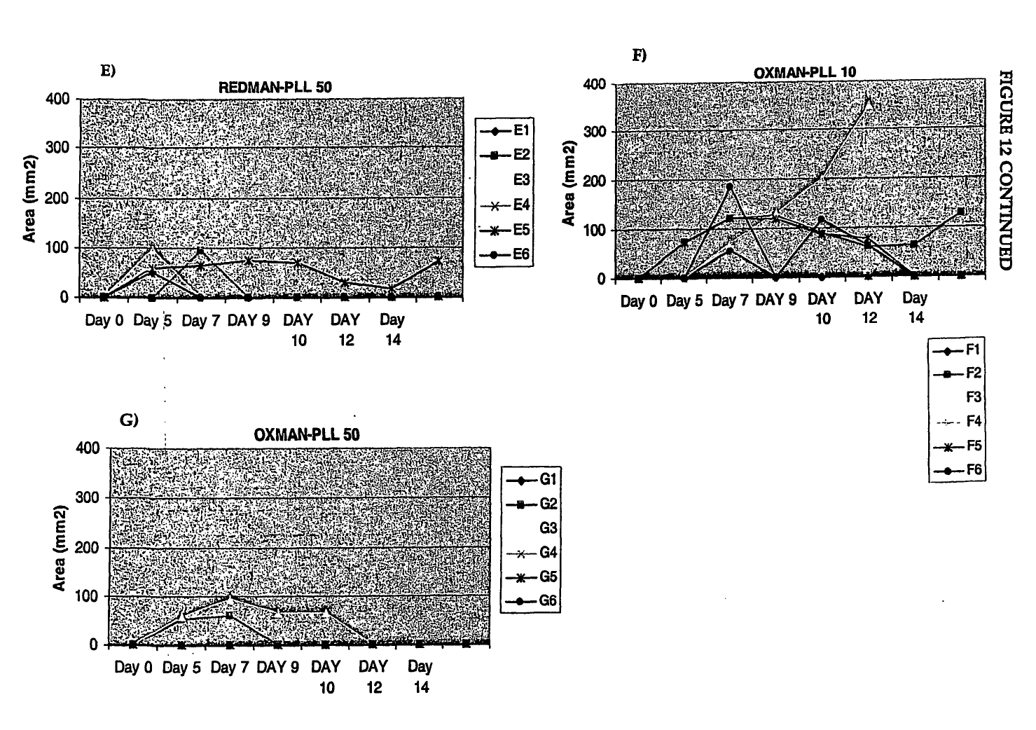

In the groups of mice treated with either PBS, PLL-DNA (10 μg) or PLL-DNA (50 μg), the EG7 cells grew to the maximal tumour size and were then rejected. In contrast, only 2/6 tumours grew to a lower maximal size in the group of mice treated with OxMan-PLL-DNA (50 μg). In the group of mice treated with RedMan-PLL-DNA (50 μg), 5/6 mice grew tumours to half the maximal size of the control groups. The OxMan-PLL-DNA (10 μg) and RedMan-PLL-DNA (10 μg) showed some therapeutic effects but were not as good as the higher doses. Results are shown graphically in FIG. 12.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Akkina, R et al, siRNAs, ribozymes and RNA decoys in modelling stem cell-based gene therapy for HIV/AIDS, Anticancer Res, 23(3A):1997 (2003).
2. Apostolopoulos, V et al, New Trends in the development of a breast cancer vaccine, Cancer Forum, 17:11 (1993).
3. Apostolpoulos, V et al, Oxidative/reductive conjugation of amnnan to antigen selects for T1 or T2 immune responses, Proc Nalt Acad Sci USA, 92:10128 (1995).
4. Apostolpoulos, V et al, Cell-mediated immune responses to MUC1 fusion protein coupled to mannan, Vaccine, 14:930 (1996).
5. Apostolpoulos, V et al, Aldehyde-mannan antigen complexes target the MHC class I antigen-presenting pathway, Eur J Immunol, 30:1714 (2000)
6. Bobek, L A et al, Molecular cloning sequence and specificity of expression of the gene encoding the low molecular weight human salivary mucin (MUC7), J Biol Chem, 268:20563 (1993).
7. Boussif, O et al, A versatile vector for gene and oligonucleotide transfer into cells n culture and in vivo: polyethylenimine, Proc Natl Acad Sci USA, 92:7297 (1995).
8. Crocker, G and Price, M R, Genetic polymorphism of high molecular weight glycoproteins: A comparative study in normal individuals and breast cancer patients, Br J Cancer, 55:651 (1987).
9. Cui, Z and Mumper, R J, Topical immunisation using nanoengineered genetic vaccines, J Control Release, 81:173 (2002a).
10. Cui, Z and Mumper, R J, Genetic immunisation using nanoparticles engineered from microemulsion precursors, Pharm Res, 19:939 (2002b).
11. Cui, Z et al, Physical characterisation and macrophage cell uptake of mannan-coated nanoparticles, Drug Dev Ind Pharm, 29:689 (2003).
12. Diebold, S S et al, Mannose polyethylenimine conjugates for targeted delivery into dendritic cells, J Biol Chem, 274:19807 (1999a).
13. Diebold, S S et al, Efficient gene delivery into human dendritic cells by adenovirus polyethylenimine and mannose polyethylenimine transfection, Hum Gene Ther, 10:775 (1999b).
14. Erbacher, P et al, Gene transfer by DNA/glycosylated polylysine complexes into human blood monocyte-derived macrophages, Hum Gene Ther, 7:721 91996).
15. Fajac, I et al, Targeting of cell receptors and gene transfer efficiency: a balancing act, Gene Ther, 9:740 (2002).
16. Ferkol, T et al, Receptor-mediated gene transfer into macrophages, Proc Natl Acad Sci USA, 93:101 (1996).
17. Liu, G et al, Biological properties of poly-L-lysine-DNA complexes generated by cooperative binding of the polycation, J Biol Chem, 276:34379 (2001).
18. Lofthouse, S A et al, Induction of T1 (cytotoxic lymphocyte) and/or T2 (antibody) responses to a mucin-1 tumour antigen, Vaccine, 15:1586 (1997).
19. Marjolijn, J L et al, A carcinoma-associated mucin is generated by a polymorphic gene encoding splice variants with alternating amino termini, J Biol Chem, 265:5573 (1990).
20. McKenzie, I F et al, Oxidised mannan antigen conjugates preferentially stimulate t1 type immune responses, Vet Immunol Immunopathol, 63:185 (1998).
21. Sato, A et al, Enhanced gene transfection in macrophages using amnnosylated cationic liposome-polyethylenimine-plasmid DNA complexes, J Drug Target, 9:201 (2001).

1-49. (canceled)

50. A compound comprising a conjugate of;
(i) a polynucleotide or oligonucleotide molecule;
(ii) a carrier comprising at least one aldehyde group; and, optionally,
(iii) a suitable linker molecule conjugating said polynucleotide or oligonucleotide with said carrier.

51. The compound of claim 50, wherein the polynucleotide or oligonucleotide molecule is an oligonucleotide molecule in the range of 5 to 50 bases in length.

52. The compound of claim 50, wherein the polynucleotide or oligonucleotide molecule is a polynucleotide molecule in the range of 50 bases to 10 kilobases in length.

53. The compound of claim 52, wherein the polynucleotide molecule is in the range of 1 to 6 kilobases in length.

54. The compound of claim 50, wherein the said polynucleotide or oligonucleotide molecule comprises an expression cassette comprising a suitable promoter sequence operably linked to a nucleotide sequence encoding a protein(s) or peptide(s).

55. The compound of claim 54, wherein said protein(s) or peptide(s) is an antigen or comprises one or more epitopes.

56. The compound of claim 54, wherein said protein(s) or peptide(s) is a polytope peptide.

57. The compound of claim 54, wherein said protein(s) or peptide(s) is an enzyme, receptor or hormone.

58. The compound of claim 50 wherein the polynucleotide or oligonucleotide molecule is an antisense RNA, catalytic RNA or small interfering RNA (siRNA).

59. The compound of claim 50 wherein the carrier comprises a plurality of aldehyde groups ranging in number from 20 to 750.

60. The compound of claim 59, wherein the carrier comprises a plurality of aldehyde groups ranging in number from 100 to 500.

61. The compound of claim 60, wherein the carrier comprises a plurality of aldehyde groups ranging in number from 200 to 400.

62. The compound of claim 50 wherein the carrier is any ligand which is recognised by a cell-surface receptor and, following binding to the receptor, can be endocytosed.

63. The compound of claim 62, wherein the carrier is a ligand selected from the group consisting of hormones, enzymes, cytokines and carbohydrate polymers.

64. The compound of claim 63, wherein the carrier is a carbohydrate polymer.

65. The compound of claim 64, wherein the carrier is an oxidised carbohydrate polymer.

66. The compound of claim 65, wherein the carrier is oxidised mannan.

67. The compound of claim 50 wherein the compound comprises a suitable linker molecule conjugating the polynucleotide or oligonucleotide molecule to the carrier.

68. The compound of claim 67, wherein the linker molecule is a polycation linker.

69. The compound of claim 68, wherein the linker molecule is selected from the group consisting of poly-L-lysine (PLL), polyethylimine (PEI), dendrimers and cationic lipids.

70. A method for cell-specific delivery of a polynucleotide or oligonucleotide molecule to a target cell(s) of a subject, said method comprising:
administering the compound of claim 50 to said subject.

71. A method for inducing an immune response to an antigen or epitope(s), wherein said immune response is primarily a CD8⁺ type of immune response, said method comprising:
providing a compound comprising a conjugate of;
(i) a polynucleotide or oligonucleotide molecule comprising a nucleotide sequence encoding an antigen or epitope(s);
(ii) a carrier comprising at least one aldehyde group; and,
optionally,
(iii) a suitable linker molecule conjugating said polynucleotide or oligonucleotide with said carrier; and
administering said compound to said subject in an amount to induce a primarily CD8⁺ type of immune response to said antigen or epitope(s).

72. The method of claim 71, wherein the carrier comprises a plurality of aldehyde groups ranging in number from 20 to 750.

73. The method of claim 72, wherein the carrier comprises a plurality of aldehyde groups ranging in number from 100 to 500.

74. The method of claim 73, wherein the carrier comprises a plurality of aldehyde groups ranging in number from 200 to 400.

75. The method of claim 70 wherein the carrier is any ligand which is recognised by a cell-surface receptor and, following binding to the receptor, can be endocytosed.

76. The method of claim 75, wherein the carrier is a ligand selected from the group consisting of hormones, enzymes, cytokines and carbohydrate polymers.

77. The method of claim 76, wherein the carrier is a carbohydrate polymer.

78. The method of claim 77, wherein the carrier is an oxidized carbohydrate polymer.

79. The method of claim 78, wherein the carrier is oxidized mannan.

80. A method for inducing an immune response to an antigen or epitope(s), wherein said immune response is primarily a CD4⁺ type of immune response, said method comprising:
providing a compound comprising a conjugate of;
(i) a polynucleotide or oligonucleotide molecule comprising a nucleotide sequence encoding an antigen or epitope(s);
(ii) a carrier comprising reduced mannan; and, optionally,
(iii) a suitable linker molecule conjugating said polynucleotide or oligonucleotide with said carrier; and
administering said compound to said subject in an amount to induce a primarily CD4⁺ type of immune response.

81. The method of claim 70 wherein the polynucleotide or oligonucleotide molecule is an oligonucleotide molecule in the range of 5 to 50 bases in length.

82. The method of claim 70 wherein the polynucleotide or oligonucleotide molecule is a polynucleotide molecule in the range of 50 bases to 10 kilobases in length.

83. The method of claim 82, wherein the polynucleotide molecule is in the range of 1 to 6 kilobases in length.

84. The method of claim 70 wherein the said polynucleotide or oligonucleotide molecule comprises an expression cassette comprising a suitable promoter sequence operably linked to a nucleotide sequence encoding a protein(s) or peptide(s).

85. The method of claim 84, wherein said protein(s) or peptide(s) is an antigen or comprises one or more epitopes.

86. The method of claim 84, wherein said protein(s) or peptide(s) is a polytope peptide.

87. A compound comprising a conjugate of;
(i) a polynucleotide or oligonucleotide molecule;
(ii) a carrier comprising reduced mannan; and, optionally,
(iii) a suitable linker molecule conjugating said polynucleotide or oligonucleotide with said carrier.

88. The compound of claim 87, wherein the polynucleotide or oligonucleotide molecule is an oligonucleotide molecule in the range of 5 to 50 bases in length.

89. The compound of claim 87, wherein the polynucleotide or oligonucleotide molecule is a polynucleotide molecule in the range of 50 bases to 10 kilobases in length.

90. The compound of claim 89, wherein the polynucleotide molecule is in the range of 1 to 6 kilobases in length.

91. The compound of claim 87 wherein the said polynucleotide or oligonucleotide molecule comprises an expression cassette comprising a suitable promoter sequence operably linked to a nucleotide sequence encoding a protein(s) or peptide(s).

92. The compound of claim 91, wherein said protein(s) or peptide(s) is an antigen or comprises one or more epitopes.

93. The compound of claim 91, wherein said protein(s) or peptide(s) is a polytope peptide.

94. The compound of claim 91, wherein said protein(s) or peptide(s) is an enzyme, receptor or hormone.

95. The compound of claim 87 wherein the polynucleotide or oligonucleotide molecule is an antisense RNA, catalytic RNA or small interfering RNA (siRNA).

96. The compound of claim 87 wherein the compound comprises a suitable linker molecule conjugating the polynucleotide or oligonucleotide molecule to the carrier.

97. The compound of claim 96, wherein the linker molecule is a polycation linker.

98. The compound of claim 97, wherein the linker molecule is selected from the group consisting of poly-L-lysine (PLL), polyethylimine (PEI), dendrimers and cationic lipids.


SEQUENCE LISTING (2 sequences)

SEQ ID NO: 1 - protein, 8 aa, Artificial sequence
  ovalbumin conjugated to mammalian CD8 epitope
SIINFEKL

SEQ ID NO: 2 - protein, 17 aa, Artificial sequence
  ovalbumin conjugated to mammalian CD4 epitope
ISQAVHAAHAEINEAGR